(19) United States
(12) Patent Application Publication
(10) Pub. No.: US 2023/0117405 A1
(43) Pub. Date: Apr. 20, 2023

(54) SYSTEMS AND METHODS FOR EVALUATION OF CHROMOSOMAL INSTABILITY USING MACHINE-LEARNING

(71) Applicants: Volastra Therapeutics, Inc., New York, NY (US); Center for Technology Licensing at Cornell University (CTL), Ithaca, NY (US)

(72) Inventors: Akanksha VERMA, New York, NY (US); Olivier ELEMENTO, New York, NY (US); Zhuoran XU, New York, NY (US)

(73) Assignees: Volastra Therapeutics, Inc., New York, NY (US); Center for Technology Licensing at Cornell University (CTL), Ithaca, NY (US)

(21) Appl. No.: 17/949,871

(22) Filed: Sep. 21, 2022

Related U.S. Application Data
(60) Provisional application No. 63/247,159, filed on Sep. 22, 2021.

(51) Int. Cl. G06T 7/00; G06T 7/11; G06V 10/764; G16H 20/00; G16H 50/20; G01N 33/574

(52) U.S. Cl. CPC G06T 7/0012; G01N 33/574; G06T 7/11; G06V 10/764; G16H 20/00; G16H 50/20; G06T 2207/20081; G06T 2207/30096

(57) ABSTRACT

The present application provides methods and systems for detecting and quantifying chromosomal instability from histology images with machine learning. Also described herein are methods for selecting treatments for a medical disease, by determining a chromosomal instability pathological metric from histology images. The disclosed methods and systems may also be used to investigate disease progression and prognosis.

### CROSS REFERENCE TO RELATED APPLICATION

This application claims priority benefit of from U.S. Provisional Application No. 63/247,159, filed on Sep. 22, 2021, the contents of which are incorporated herein by reference in its entirety.

### FIELD

The present application relates to the methods and systems for determining chromosomal instability in a biological image, and use of the determined chromosomal instability for disease treatment and diagnosis.

### BACKGROUD

Chromosomal instability (CIN) results from errors in chromosome segregation during mitosis and contributes to poor prognosis, metastasis, and therapeutic resistance in human cancers. Identifying patient tumors with high levels of CIN can improve outcomes because CIN specific treatment options are available. Current methods to detect CIN in patient samples, such as bulk DNA sequencing, evaluation of whole slide histological images are not high throughput enough to be standard of care in the clinic. Whole slide histology images and/or genomic sequencing of tumors are often acquired during tumor extraction. Previous high throughput methods have used machine-learning techniques to identify cells undergoing mitosis in histological images. See, for example, Al-Janabi et al. 2013. “Evaluation of Mitotic Activity Index in Breast Cancer Using Whole Slide Digital Images”, PLOS ONE, 8(12):e82576. However, these methods have not been able predict the level of CIN in the images. In order to better diagnose and treat cancer patients there is a need to detect CIN from readily available histology images with high accuracy and efficiency. The present disclosure addressed these and other needs.

### BRIEF SUMMARY

Chromosomal instability refers to ongoing chromosome segregation errors throughout consecutive cell divisions that may result in various pathological phenotypes including copy number gains or losses (e.g., altered genome), increased copy number heterogeneity, increased presence of micronuclei, elevated expression of cyclic GMP-AMP synthase (cGAS)-stimulator of interferon genes (STING) activity, and/or CIN23 gene signature. Elevated chromosomal instability, resulting in various instances of chromosomal instability pathology, has been correlated with poor prognosis in several cancers. Thus, the present application provides machine-learning models to analyze images of biological samples to characterize chromosomal instability in a patient sample (e.g., a tumor resection sample) using high throughput machine-learning models.

In one aspect, disclosed herein are methods and systems to train a machine-learning model with training histological images and matched chromosomal instability pathological metric corresponding to the training histological images, to predict the pathological status of the input histological images and to output a pathological status metric to the user. In some embodiments, the pathological status metric is a probability of high chromosomal instability in the image, a continuous chromosomal instability score, or a binary classification of high or low chromosomal instability. Further, provided herein are methods and systems for characterizing a disease (e.g., cancer) in a patient from one or more histological images using a machine-learning model trained according to the methods of the present disclosure. The systems and methods can be applied, for example to a diagnostic or prognostic method, used to inform treatment selection (e.g., selection of a pharmaceutical drug), and/or evaluate the efficacy of a treatment, to further characterize a disease (e.g., cancer) in a patient.

Thus, in some aspects, provided herein is a method for characterizing a disease in a patient, comprising: inputting one or more input histological images of a biological sample into a machine-learning model, wherein the machine learning model is trained using a plurality of training histological images and one or more matched chromosomal instability pathological metrics corresponding to the plurality of training histological images; and, classifying a pathological status of the biological sample in the one or more input histological images using the trained machine-learning model.

In some embodiments according to the method described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the methods described above, the matched chromosomal pathological metric is obtained from DNA from the biological sample of the training histological image. In some embodiments, the matched chromosomal pathological metric is computed from DNA from the same patient as the biological sample of the training histological image. In some embodiments, the matched chromosomal pathological metric and the biological sample of the training histological image come from the same patient.

In some embodiments according to any of the methods described above, the one or more input histological images and/or the plurality of training histological images is an image captured between 2.5 x and 20 x magnification. In some embodiments, the one or more input histological images and/or the plurality of training histological images is captured at a resolution between 256 pixel x 256 pixel and 10,000 pixel x 10,000 pixel. In some embodiments, the one or more input histological images and/or the plurality of training histological images are hematoxylin and eosin (H&E) and/or 4′,6-diamidino-2-phenylindole (DAPI) stained images.

In some embodiments according to any of the methods described above, the method further comprises segmenting one or more whole images into a plurality of image tiles, wherein the image tiles are inputted into the machine-learning model as the input histological images and/or the training histological images.

In some embodiments according to any of the methods described above, the machine-learning model segments the input histological images and/or the training histological images into tiles.

In some embodiments according to any of the methods described above, one or more of the input histological images and/or the plurality of training images are deposited into computer cloud storage.

In some embodiments according to any of the methods described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

In some embodiments according to any of the methods described above, the matched chromosomal instability pathological metric and the training histological images are used to predict the pathological status of the input histological images. In some embodiments, the pathological status is described as a metric, wherein the pathological status metric is selected from the group consisting of a probability of high chromosomal instability in the image, a continuous chromosomal instability score, and a binary classification of high or low chromosomal instability. In some embodiments, the pathological status metric is displayed to a user. In some embodiments, the matched chromosomal instability pathological metrics are displayed to a user.

In some embodiments according to any of the methods described above, characterizing a disease comprises diagnosing the disease. In some embodiments, characterizing a disease comprises informing a treatment strategy. In some embodiments, characterizing a disease comprises evaluating the disease progression. In some embodiments, characterizing a disease comprises predicting the disease prognosis. In some embodiments, characterizing a disease comprises evaluating effect of a treatment. In some embodiments, characterizing a disease comprises identifying a patient population for treatment. In some embodiments, the disease is a cancer.

In some embodiments according to any of the methods described above, the method is implemented on a cloud-based computing platform.

In other aspects, provided herein is a system for characterizing a disease in a patient with machine-learning, comprising: one or more processors; a memory; and one or more programs with instructions for: receiving data representing one or more input histological images of a biological sample; and, classifying a pathological status of the biological sample in the one or more input histological images using a trained machine-learning model trained using a plurality of training histological images and one or more matched chromosomal instability pathological metrics corresponding to the plurality of training histological images.

In some embodiments according to the system described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the systems described above, the matched chromosomal pathological metric is obtained from DNA from the biological sample of the training histological image. In some embodiments, the matched chromosomal pathological metric is computed from DNA from the same patient as the biological sample of the training histological image. In some embodiments, the matched chromosomal pathological metric and the biological sample of the training histological image come from the same patient.

In some embodiments according to any of the systems described above, the one or more input histological images and/or the plurality of training histological images is an image captured between 2.5 x and 20 x magnification. In some embodiments, the one or more input histological images and/or the plurality of training histological images is captured at a resolution between 256 pixel x 256 pixel and 10,000 pixel x 10,000 pixel. In some embodiments, the one or more input histological images and/or the plurality of training histological images are hematoxylin and eosin (H&E) and/or 4′,6-diamidino-2-phenylindole (DAPI) stained images.

In some embodiments according to any of the systems described above, the instructions further comprise instructions for segmenting one or more whole images into a plurality of image tiles, wherein the image tiles are inputted into the machine-learning model as the input histological images and/or the training histological images.

In some embodiments according to any of the systems described above, the machine-learning model segments the input histological images and/or the training histological images into tiles.

In some embodiments according to any of the systems described above, the one or more input histological images and/or the plurality of training histological images are deposited into a computer cloud.

In some embodiments according to any of the systems described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

In some embodiments according to any of the systems described above, the matched chromosomal instability pathological metric and training histological images are used to predict the pathological status of the input histological images. In some embodiments, the pathological status is described as a metric, wherein the pathological status metric is selected from the group consisting of a probability of high chromosomal instability in the image, a continuous chromosomal instability score, and a binary classification of high or low chromosomal instability. In some embodiments, the pathological status metric is displayed to a user. In some embodiments according to any of the systems described above, the matched chromosomal instability pathological metrics are displayed to a user.

In some embodiments according to any of the systems described above, characterizing a disease comprises diagnosing the disease. In some embodiments, characterizing a disease comprises informing a treatment strategy. In some embodiments, characterizing a disease comprises evaluating the disease progression. In some embodiments, characterizing a disease comprises predicting the disease prognosis. In some embodiments, characterizing a disease comprises evaluating effect of a treatment. In some embodiments, characterizing a disease comprises identifying a patient population for treatment. In some embodiments, the disease is a cancer.

In some embodiments according to any of the systems described above, the instructions are implemented on a cloud-based computing platform. In some embodiments, the instructions for implementing the instructions reside in cloud storage.

In other aspects, provided herein is a method for training a machine-learning model to analyze histological images of biological samples, comprising: obtaining a chromosomal instability pathological metric for each training histological image of a plurality of training histological images; and training the machine-learning model based on the plurality of training histological images and the matched chromosomal instability pathological metrics, wherein the machine-learning model is trained to receive one or more input histological images and output a pathological status of the one or more input histological image.

In some embodiments according to the method of training described above, the biological samples comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the methods of training described above, the matched chromosomal pathological metric is obtained from DNA from the biological sample of the training histological image. In some embodiments, the matched chromosomal pathological metric is computed from DNA from the same patient as the biological sample of the training histological image. In some embodiments, the matched pathological metric and the biological sample of the training histological image come from the same patient.

In some embodiments according to any of the methods of training described above, the one or more input histological images and/or the plurality of training histological images is an image captured between 2.5 x and 20 x magnification. In some embodiments, the one or more input histological images and/or the plurality of training histological images is captured at a resolution between 256 pixel x 256 pixel and 10,000 pixel x 10,000 pixel. In some embodiments, the one or more input histological images and/or the plurality of training histological images are hematoxylin and eosin (H&E) and/or 4′,6-diamidino-2-phenylindole (DAPI) stained images.

In some embodiments according to any of the methods of training described above, the matched chromosomal instability pathological metric describes the chromosomal instability of the biological sample. In some embodiments, the matched chromosomal instability pathological metric is a fraction of genome altered. In some embodiments, the fraction of genome altered is calculated using DNA sequencing data.

In some embodiments according to any of the methods of training described above, the method further comprises segmenting one or more whole images into a plurality of image tiles, wherein the image tiles are inputted into the machine-learning model as the input histological images and/or the training histological images.

In some embodiments according to any of the methods of training described above, the machine-learning model segments the input histological images and/or the training histological images into tiles.

In some embodiments according to any of the methods of training described above, the one or more of the input histological images and/or the plurality of training histological images are deposited into a computer cloud.

In some embodiments according to any of the methods of training described above, the machine-learning model is an unsupervised model. In some embodiments, the machine-learning model is a weakly- supervised model. In some embodiments, the machine-learning model is a human-in-the-loop model. In some embodiments, the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

In some embodiments according to any of the methods of training described above, the matched chromosomal instability pathological metric and training histological images are used to predict the pathological status of the input histological images. In some embodiments, the pathological status metric is selected from the group consisting of a probability of high chromosomal instability in the image, a continuous chromosomal instability score, and a binary classification of high or low chromosomal instability. In some embodiments, the pathological status metric is displayed to a user. In some embodiments, the matched chromosomal instability pathological metrics are displayed to a user.

In some embodiments according to any of the methods of training described above, the method is implemented on a cloud-based computing platform.

In other aspects, provided herein is a system for training a machine-learning model to predict a pathological status, comprising one or more processors; a memory; and one or more programs with instructions for: receiving a plurality of chromosomal instability pathological metrics for a plurality of training histological images by calculating the chromosomal instability pathological metrics in the plurality of training histological images; training the machine-learning model based on the plurality of training histological images and the matched chromosomal instability pathological metrics, wherein the machine-learning model is trained to receive one or more input histological images and output a pathological status of the one or more input histological images.

In some embodiments according to the system of training described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the systems of training described above, the matched chromosomal pathological metric is obtained from DNA from the biological sample of the training histological image. In some embodiments, the matched chromosomal pathological metric is computed from DNA from the same patient as the biological sample of the training histological image. In some embodiments, the matched chromosomal pathological metric and the biological sample of the training histological image come from the same patient.

In some embodiments according to any of the systems of training described above, the one or more input histological images and/or the plurality of training histological images is an image captured between 2.5 x and 20 x magnification. In some embodiments, the one or more input histological images and/or the plurality of training histological images is captured at a resolution between 256 pixel x 256 pixel and 10,000 pixel x 10,000 pixel. In some embodiments, the one or more input histological images and/or the plurality of training histological images are hematoxylin and eosin (H&E) and/or 4′,6-diamidino-2-phenylindole (DAPI) stained images.

In some embodiments according to any of the systems of training described above, the matched chromosomal instability pathological metric describes the chromosomal instability of the biological sample. In some embodiments, the matched chromosomal instability pathological metric is a fraction of genome altered. In some embodiments, the fraction of genome altered is calculated using DNA sequencing data.

In some embodiments according to any of the systems of training described above, the instructions further comprise instructions for segmenting one or more whole images into a plurality of image tiles, wherein the image tiles are inputted into the machine-learning model as the input histological images and/or the training histological images.

In some embodiments according to any of the systems of training described above, the machine-learning model segments the input histological images and/or the training histological images into tiles.

In some embodiments according to any of the systems of training described above, the one or more input histological images and/or the plurality of training histological images are deposited into a computer cloud.

In some embodiments according to any of the systems of training described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from Support Vector Machines (SVM), Random Forests(RF), Artificial Neural Network (ANN), Convolutional Neural Network (CNN), K-means, ResNet, DenseNet, and eXtreme Gradient Boosting (XGBoost).

In some embodiments according to any of the systems of training described above, the matched chromosomal instability pathological metrics and training histological images are used to predict the pathological status of the input histological image. In some embodiments, the pathological status is described as a metric, wherein the pathological status metric is selected from the group consisting of a probability of high chromosomal instability in the image, a continuous chromosomal instability score, and a binary classification of high or low chromosomal instability. In some embodiments, the pathological status metric is displayed to a user. In some embodiments, the matched chromosomal instability pathological metrics are displayed to a user.

In some embodiments according to any of the systems of training described above, the instructions are implemented on a cloud-based computing platform.

In other aspects, provided herein is a non-transitory computer-readable storage medium storing one or more programs, the one or more programs comprising instructions, which when executed by one or more processors of an electronic device having a display, cause the electronic device to: receive one or more input histological images of a biological sample; input one or more input histological images of a biological sample into a machine-learning model trained using a plurality of training histological images and one or more matched chromosomal instability pathological metrics corresponding to the plurality of training histological images; and, classify a pathological status of the biological sample in the one or more input histological images using the training machine-learning model.

In some embodiments according to the computer-readable storage medium described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the computer-readable storage mediums described above, the matched chromosomal pathological metric is obtained from DNA from the biological sample of the training histological image. In some embodiments, the matched chromosomal pathological metric is computed from DNA from the same patient as the biological sample of the training histological image. In some embodiments, the matched chromosomal pathological metric and the biological sample of the training histological image come from the same patient.

In some embodiments according to any of the computer-readable storage mediums described above, the one or more input histological images and/or the plurality of training histological images is an image captured between 2.5 x and 20 x magnification. In some embodiments, the one or more input histological images and/or the plurality of training histological images is captured at a resolution between 256 pixel x 256 pixel and 10,000 pixel x 10,000 pixel. In some embodiments, the one or more input histological images and/or the plurality of training histological images are hematoxylin and eosin (H&E) and/or 4′,6-diamidino-2-phenylindole (DAPI) stained images.

In some embodiments according to any of the computer-readable storage mediums described above, the matched chromosomal instability pathological metric describes the chromosomal instability of the biological sample. In some embodiments, the matched chromosomal instability pathological metric is a fraction of genome altered. In some embodiments, the fraction of genome altered is calculated using DNA sequencing data.

In some embodiments according to any of the computer-readable storage mediums described above, the instructions further comprise instructions for segmenting one or more whole images into a plurality of image tiles, wherein the image tiles are inputted into the machine-learning model as the input histological images and/or the training histological images.

In some embodiments according to any of the computer-readable storage mediums described above, the machine-learning model segments the input histological images and/or the training histological images into tiles.

In some embodiments according to any of the computer-readable storage mediums described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

In some embodiments according to any of the computer-readable storage mediums described above, the matched chromosomal instability pathological metrics and training histological images are used to predict the pathological status of the one or more input histological images. In some embodiments, the pathological status is described as a metric, wherein the pathological status metric is selected from the group consisting of a probability of high chromosomal instability in the image, a continuous chromosomal instability score, and a binary classification of high or low chromosomal instability. In some embodiments, the pathological status metric is displayed to a user. In some embodiments, the matched chromosomal instability pathological metrics are displayed to a user.

In some embodiments according to any of the computer-readable storage mediums described above, the one or more computer programs are implemented on a cloud-based computing platform. In some embodiments, the instructions for implementing the one or more computer programs reside in cloud storage.

### BRIEF DESCRIPTION OF THE DRAWINGS

Various aspects of the disclosed methods and systems are set forth with particularity in the appended claims. A better understanding of the features and advantages of the disclosed methods and systems will be obtained by reference to the following detailed description of illustrative embodiments and the accompanying drawings, of which:

FIG. 1 illustrates a flowchart for training the machine-learning model according to examples of the disclosure.

FIG. 2 illustrates an exemplary process for segmenting a histological image (e.g., a training histological image) of a patient primary tumor or metastatic tumor in a plurality of training histological image tiles used to train the machine-learning model according to examples of the disclosure.

FIG. 3 illustrates a method for pre-training a machine-learning model to diagnose a patient with a high chromosomal instability tumor, stratify patients based on chromosomal instability metrics, or recommend chromosomal instability specific treatments according to examples of the disclosure.

FIG. 4 illustrates a computing system according to examples of the disclosure.

FIG. 5 illustrates a computing system comprising a cloud according to examples of the disclosure.

FIG. 6 shows an exemplary output of a machine-learning model of the present disclosure.

### DETAILED DESCRIPTION

The present application provides methods and systems to characterize chromosomal instability in a tumor sample using a high throughput machine-learning approach. Chromosomal instability describes the rate of abnormal cell division, mitosis, in a tumor sample. Chromosomal instability can be inferred using a number of approaches such as measuring gene expression of immune pathways, calculating the percent of the genome altered with DNA sequencing, visual detection of micronuclei, or any combination thereof. Because chromosomal instability has been linked to cancer stage, cancer prognosis, and probability of cancer treatment success, the disclosed methods and systems can help improve disease outcomes, and enable the discovery of new and novel therapeutic targets. Ultimately, these improvements may heighten the standard of care for patients with cancer.

Disclosed herein are method and systems to train a machine-learning model with training histological images (e.g., histological images of a biological sample, optionally wherein the biological sample comprises a tumor sample) and one or more matched chromosomal instability pathological metrics to classify the pathological status of a biological sample (e.g., the biological sample of the training histological image). In some embodiments, a pathological status metric is outputted to a user. In some embodiments, the pathological status is described as a pathological status metric related to chromosomal instability of the biological sample of the training histological images. Exemplary chromosomal instability pathological metrics include, but are not limited to bulk genomic aneuploidy burden, fraction of the genome altered (FGA), cyclic GMP-AMP synthase (cGAS)-stimulator of interferon genes (STING) activity, CIN23 gene signature, increased copy number heterogeneity, gain/loss of chromosomes, type I interferon expression, increased presence of micronuclei, etc. The chromosomal instability pathological metric can be evaluated using any suitable technique known in the art, wherein the technique is related to the specific metric to be determined.

The training histological images may be segmented into training histological image tiles. Each tile may be a segment of a whole training histological image. In some embodiments, the training histological images are segmented into a plurality of training histological image tiles prior to inputting the one or more training histological images into the machine-learning model.

Accordingly, one aspect of the present application provides methods and systems to train a machine-learning model. The machine-learning model may be trained with a plurality of training histological images, or training histological image tiles that are segments of a whole training histological image, and a matched chromosomal instability pathological metric, such as any of the chromosomal instability pathological metrics of the present disclosure. The training histological images and matched chromosomal instability pathological metrics may be obtained from a publicly available database. In some embodiments, the machine-learning model is trained with publicly available histological images of tumor samples. The histological images may be obtained from any source of histological images. In some embodiments, the training histological images of tumor samples are not publicly available images (e.g., the training histological images are obtained from a private source, such as sourced from internal collaborations, licensed data, a clinic, a hospital system, a company or any other entity with access to histological images). In some embodiments, the training histological images of tumor samples are publicly available images. The training histological images may be downloaded from a publicly available database. In some embodiments, the publicly available database may comprise images of one or more types of tumors.

In some embodiments, the training histological images or training histological tiles are used to train a machine-learning model. In some embodiments, the machine-learning model is an unsupervised machine-learning model. In some embodiments, the training histological images or training histological tiles are used to train a weakly-supervised machine-learning model. In some embodiments, the training histological images or training histological tiles are used to train a human-in-the-loop machine-learning model. The machine-learning model, such as any of the machine-learning models described herein, may be trained with training histological images and matched chromosomal instability pathological metrics as described herein.

The machine-learning model may apply a Support Vector Machines (SVM), Random Forests (RF), Artificial Neural Network (ANN), Convolutional Neural Network (CNN), K-means, ResNet, DenseNet or eXtreme Gradient Boosting (XGBoost) model. The machine-learning model may apply any additional model known in the art or described herein.

A chromosomal instability pathological metric matched to a training histological image may relate to the same biological sample as the training histological image and may be computed from DNA sequencing, RNA sequencing, or protein quantification. The chromosomal instability pathological metric may be any metric known in the art to infer chromosomal instability from a biological sample.

In some embodiments, the present application provides a method for training a machine-learning model to analyze histological images (e.g., input histological images) of biological samples. Once the machine-learning model is trained, a physician, pathologist, or other medical provider can prepare a patient biological sample for input into the machine-learning model. The biological sample may be a tumor sample stained with hematoxylin and eosin (H&E) or 4′,6-diamidino-2-phenylindole (DAPI), that may be fixed onto a slide for histological imaging. The histological image (e.g., input histological image) may be segmented into tiles (e.g., input histological image tiles) and are inputted into the trained machine-learning model. In some embodiments, the input histological images are segmented into a plurality of input histological image tiles prior to inputting the one or more input histological images into the machine-learning model. The segmenting may be implemented as part of the machine-learning model. The machine-learning model may predict a pathological status metric, such as a level of chromosomal instability, for each input histological image tile. The results from each tile may be aggregated to prepare (e.g., compute) a pathological status metric, related to chromosomal instability, corresponding to the whole input histological image. The pathological status metric can be used to better characterize the pathology of the patient’s biological sample, and inform characterization of a disease in the patient.

Another aspect of the present application provides a method for characterizing a disease in a patient, using a machine-learning model configured to analyze histological images of biological samples. In some embodiments, the characterization of the disease may comprise diagnosing the disease, informing a treatment strategy, evaluating the disease progression, predicting the disease prognosis, evaluating effect of a treatment, identifying a patient population for treatment, or any combination thereof. In some embodiments, the disease is a cancer.

In some embodiments, the biological sample from the patient comprise a slice of a biological tissue sample that has been biopsied for use in the disclosed methods or for other diagnostics, surveillance, or treatment procedures. The biological tissue sample may be a biopsy of a solid tumor. One or more histology slides may be prepared from the biological sample as described herein. The tissue may be stained with H&E or DAPI, or another tissue stain used in the art, as described herein.

In some embodiments, an input histological image is an image captured of the prepared histology slide. The image may be captured at a resolution ranging from 256 pixel x 256 pixel to 10,000 pixel x 10,000 pixel. The resolution may depend on the magnification of the histological image. In some embodiments, the magnification is varied within a histological image or histological image tile to ensure the determination of a pathological status. The image may be captured between about 2.5 x and about 20 x magnification. The input histological image may be a plurality of captured segmented image tiles from the same slide. In some embodiments, the input histological image is segmented into input histological image tile prior to inputting the input histological image into the machine-learning model. In some embodiments, the whole input histological image is inputted into the machine-learning model and the model segments the whole input histological image into input histological image tiles. In some embodiments, the input histological image, plurality of input histological images, and/or image tiles thereof, are deposited into computer cloud storage.

In some embodiments, the input histological image or plurality of input histological image tiles are inputted into a trained machine-learning model, wherein the machine-learning model has been trained with a plurality of training histological images, or image tiles thereof, and matched chromosomal instability pathological metrics. The machine-learning model may be trained to classify the pathological status of the biological sample represented in the input histological image or input histological image tiles. The pathological status may relate to the level of chromosomal instability in the input histological image or plurality of input histological image tiles. The pathological status may be displayed to a user. In some embodiments, the pathological status is described as a metric, wherein the pathological status metric is selected from the group consisting of a probability of high chromosomal instability in the image, a continuous chromosomal instability score, and a binary classification of high or low chromosomal instability. The pathological status metric may be displayed to a user. In some embodiments, the pathological status (e.g., the pathological status metric) may be used to diagnose a patient, inform a treatment strategy, evaluate disease progression, predict the disease prognosis, evaluate effect of a treatment, identify if the patient is part of a larger patient population, or for any other application in the art. For example, if a patient’s biological sample has a high chromosomal instability metric corresponding to a high fraction of the genome altered, a physician may prescribe an anti-chromosomal instability therapeutic as described herein.

This application will first provide high-level descriptions for the disclosed methods and systems, including training the disclosed machine-learning model and using the training machine-learning model to characterize a disease in a patient. Second, the application will provide key definitions. Third, the application will describe the samples and sample preparation methods to implement the methods and systems described herein. Fourth, the application will describe potential image processing methods. Fifth, the application will describe the data that may be used to train the machine-learning model. Sixth, the application will describe the machine-learning models, models, and statistical data analysis methods that may be used to carry out the methods and systems disclosed herein. Finally, the application provides example use cases for the methods and systems described herein.

### I. Definitions

Although the following description uses terms “first,” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

The term “chromosomal instability”, “chromosome instability”, or “CIN” refers to chromosome segregation errors throughout consecutive mitotic events and comprises both structural, numerical, genomic, and pathological CIN as used in the art.

The term “image feature” refers to a property of an image or image patch that contains information about the content of the image or image patch, e.g., image features may be specific structures in the image or image patch such as points, edges, shapes, textures, or objects, or may be non-visual or non-human-interpretable properties of the image derived from an image processing- and/or machine learning-based analysis of an image.

As used herein, the terms “classification model” and “classifier” are used interchangeably, and refer to a machine learning architecture or model that has been trained to sort input data into one or more labeled classes or categories.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease or condition, delay in recurrence of the disease or condition, or inhibition of the disease or condition. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

As used herein, the term “cloud” refers to shared or sharable storage of software and/or electronic data using, e.g., a distributed network of computer servers. In some instances, the cloud may be used, e.g., for archiving electronic data, sharing electronic data, and analyzing electronic data using one or more software packages residing locally or in the cloud.

The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

### II. Samples and Sample Preparation

The methods disclosed herein may be used to analyze a diverse array of samples (e.g., biological samples). In some embodiments, the biological sample comprises at least a portion of a solid tumor. The biological sample may imaged for use in the methods provided herein. In some embodiments, the biological sample is stained, and subsequently imaged, for use in the methods provided herein.

A sample disclosed herein can be or can be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In some embodiments, the sample includes a tumor sample.

Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy for said disease. In some embodiments, the biological sample is from an individual subject. In some embodiments, the individual subject is a mammal, such as a human, bovine, horse, feline, canine, rodent, or primate. In some embodiments, the method comprises obtaining the biological sample from an individual (e.g., human).

The biological sample can be obtained as a tissue sample, such as a tissue section, biopsy, a core biopsy, needle aspirate, or fine needle aspirate. In some embodiments, the sample can be a fluid sample, such as a blood sample, urine sample, or saliva sample. In some embodiments, the sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface (e.g., a substrate), such as a glass slide.

Biological samples can include one or more diseased cells. A diseased cell can have altered metabolic properties, gene expression, protein expression, and/or morphologic features, such as altered chromosome morphology and/or altered mitotic state. Examples of diseases include inflammatory disorders, metabolic disorders, nervous system disorders, and cancer.

In some embodiments, the cancer is a solid tumor or a hematologic malignancy. In some embodiments, the cancer is a carcinoma, a sarcoma, a lymphoma, or a leukemia. In some instances, the cancer is a naive cancer, or a cancer that has not been treated by a particular therapeutic agent. In some embodiments, the cancer has previously been treated by a particular therapeutic agent. In some embodiments, the cancer is a primary tumor or a primary cancer, which is a tumor that originated in the location or organ in which it is present and did not metastasize to that location from another location. In some embodiments, the cancer is a metastatic cancer. In some embodiments, the cancer is a relapsed or refractory cancer.

In some embodiments, a tumor or cancer originates from blood, lymph node, liver, brain/neuroblastoma, esophagus, trachea, stomach, intestine, colon, rectum, anus, pancreas, throat, tongue, bone, ovary, uterus, cervix, peritoneum, prostate, testes, breast, kidney, lung, or skin, gastric, colorectal, bladder, head and neck, nasopharyngeal, endometrial, bile duct, oral, multiple myeloma, leukemia, soft tissue sarcoma, gall bladder, endocrine, mesothelioma, Wilms tumor, duodenum, neuroendocrine, salivary gland, larynx, choriocarcinoma, cardial, small bowel, eye, or germ cell cancer.

In some embodiments, a cancer (e.g., a primary tumor) includes, but is not limited to, acute lymphoblastic leukemia (ALL), acute myeloid leukemia (AML), bladder cancer, breast cancer, brain cancer, cervical cancer, colon cancer, colorectal cancer, endometrial cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, prostate cancer, squamous cell carcinoma, testicular cancer, stomach cancer, or thyroid cancer. In some instances, a cancer includes a lymphoid neoplasia, head and neck cancer, pancreatic cancer, endometrial cancer, colon or colorectal cancer, prostate cancer, glioma or other brain/spinal cancers, ovarian cancer, lung cancer, bladder cancer, melanoma, breast cancer, a myeloid neoplasia, testicular cancer, stomach cancer, cervical, kidney, liver, or thyroid cancer.

In some embodiments, cancer cells can be derived from solid tumors, hematological malignancies, cell lines, organoids, or obtained as circulating tumor cells. Biological samples can also include fetal cells and immune cells. In some embodiments, the biological sample comprises at least a portion of a tumor sample. In some embodiments, the biological sample comprises cancer cells derived from solid tumors. In some embodiments, the biological sample comprises a solid tumor, or portion thereof, and is obtained from a tissue biopsy from an individual suspected of having a disease (e.g., cancer).

In some embodiments, a substrate herein can be any support that is insoluble in aqueous liquid and which allows for positioning of biological samples on the support. In some embodiments, the biological sample can be attached to a substrate. In some embodiments, the substrate facilitates the visualization of the morphology of the biological sample. In some embodiments, the sample can be attached to the substrate reversibly by applying a suitable polymer coating to the substrate, and contacting the sample to the polymer coating. In some embodiments, the sample can be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A biological sample can be harvested from a subject (e.g., via surgical biopsy or whole subject sectioning) or grown in vitro as a population of cells, and prepared for analysis as a tissue slice or tissue section. In some embodiments, the samples grown in vitro may be sufficiently thin for analysis without further processing steps. Alternatively, the samples grown in vitro, and samples obtained via biopsy or sectioning, can be prepared as thin tissue sections using a mechanical cutting apparatus such as a vibrating blade microtome. In some embodiments, a thin tissue section can be prepared by applying a touch imprint of a biological sample to a suitable substrate material.

The thickness of a tissue section typically depends on the method used to prepare the section and the physical characteristics of the tissue, and therefore sections having a wide variety of different thicknesses can be prepared and used for the methods described herein. For example, in some embodiments, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 µm. Thicker sections can also be used if desired or convenient, e.g., at least 70, 80, 90, or 100 µm or more. In some embodiments, the thickness of the tissue section can be greater than about 100, 90, 80, 70, 60, 50, 40, 30, 20, 15, 14, 13, 12, 11, 10, 9, 8, 7, 6, 5, 4, 3, 2, 1.5, 1.0, 0.7, 0.5, 0.4, 0.3, 0.2, 0.1 µm or less. In some embodiments, the thickness of a tissue section is between 1-100 µm, 1-50 µm, 1-30 µm, 1-25 µm, 1-20 µm, 1-15 µm, 1-10 µm, 2-8 µm, 3-7 µm, or 4-6 µm, however, sections with thicknesses larger or smaller than these ranges can also be analyzed using the machine-learning model described herein.

Multiple sections can also be obtained from a single biological sample. For example, in some embodiments, multiple tissue sections can be obtained from a surgical biopsy sample by performing serial sectioning of the biopsy sample using a sectioning blade. In some embodiments, spatial information of the biopsy sample is maintained through the serial sectioning process.

In some embodiments, the biological sample (e.g., a tissue section from a tumor biopsy, as described above) can be prepared by deep freezing at a temperature suitable to maintain or preserve the integrity (e.g., the physical characteristics) of the tissue structure. In some embodiments, the frozen tissue sample can be sectioned, such as thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. In some embodiments, the tissue sample is prepared at a temperature that is, for example, less than -15° C., less than -20° C., or less than -25° C.

In some embodiments, the biological sample can be prepared using formalin-fixation and paraffin-embedding (FFPE). In some embodiments, cell suspensions and other non-tissue samples can be prepared using formalin-fixation and paraffin-embedding. In some embodiments, following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples.

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material and activating the polymer material to form a hydrogel. In some embodiments, the hydrogel is formed such that the hydrogel is internalized within the biological sample.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample comprises cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 µm to about 2 mm.

Additional methods and aspects of hydrogel embedding of biological samples are described for example in Chen et al., Science 347(6221):543-548, 2015, the entire contents of which are incorporated herein by reference.

To facilitate visualization, biological samples can be stained using a wide variety of stains and staining techniques. In some embodiments, the visualization comprises visualization of micronuclei and/or chromatin. In some embodiments, the visualization comprises visualization of micronuclei.

In some embodiments, for example, a biological sample can be stained using any number of stains, including but not limited to, Feulgen, acridine orange, DAPI, eosin, ethidium bromide, and haematoxylin. In some embodiments, the sample is stained using hematoxylin and eosin (H&E) staining techniques. In some embodiments, the stained biological samples are imaged. Image processing techniques are described in Section III.A.

### III. Methods

The methods provided herein comprise methods for training a machine-learning model, and methods of characterizing a disease in a patient using a machine-learning model trained using the training methods described herein. In some embodiments, the methods include training a machine-learning model using a plurality of training histological images, or image tiles thereof, and a corresponding chromosomal instability pathological metric. In some embodiments, the chromosomal instability pathological metric is separately obtained. The chromosomal instability pathological metric may infer chromosomal instability using approaches known in the art such as measuring gene expression of immune pathways, calculating the percent of the genome altered with DNA sequencing, and visual detection of micronuclei. In some embodiments, the methods include characterizing a disease in a patient using input histological images, or image tiles thereof.

### A. Image Processing

The disclosed methods (e.g., methods of training a machine-learning model and/or methods of charactering a disease in a patient using a machine-learning model trained using the method of training a machine learning model described herein) may be utilized with images, e.g., whole slide pathology images or tiles thereof, of tissue samples that have been acquired using any of a variety of microscopy imaging techniques known to those of skill in the art. Examples of imaging techniques include, but are not limited to, bright-field microscopy, dark-field microscopy, phase contrast microscopy, differential interference contrast (DIC) microscopy, fluorescence microscopy, confocal microscopy, confocal laser microscopy, super-resolution optical microscopy, scanning or transmission electron microscopy, and the like.

The imaging techniques described herein may be used to capture training images and/or input images. In some embodiments, the training images and/or input images are histological images, such as training histological images and input histological images. In some embodiments, the imaging techniques are used to capture stained (e.g., hematoxylin and eosin (H&E) stained or 4′,6-diamidino-2-phenylindole (DAPI) stained) training histological images of a biological sample. In some embodiments, the imaging techniques are used to capture stained (e.g., H&E stained or DAPI stained) input histological images of a biological sample. In some embodiments, the biological sample comprises a tumor sample, such as at least a portion of a solid tumor (e.g., a biopsy slice of a solid tumor). In some embodiments, the biological sample comprises any of the biological samples described in Section II.

In some embodiments, the plurality of training histological images are segmented into tiles prior to imaging. In some embodiments, the plurality of training histological images are segmented into tiles after imaging. In some embodiments, the input histological image is segmented into tiles prior to imaging. In some embodiments, the input histological image is segmented into tiles after imaging. In some embodiments, the imaging is implemented at part of a machine-learning model, such as any of the machine-learning models described herein.

In some embodiments, microscopy is used to capture a digital image of the biological sample (e.g., a biological sample comprising a tumor sample). In some embodiments, the biological sample is stained (e.g., H&E stained) prior to microscopy imaging. In some aspects, bright-field microscopy is used to capture histological images of the biological sample. In bright-field microscopy, light is transmitted through the biological sample and the contrast is generated by the absorption of light in dense areas of the biological sample. In some embodiments, the biological sample is stained (e.g., H&E stained) prior to bright-field imaging.

In some aspects, fluorescence microscopy is used to capture a digital image of the biological sample (e.g., a biological sample comprising a tumor sample). In some embodiments, the biological sample is stained (e.g., H&E stained or DAPI stained) prior to fluorescence imaging. In some aspects, a fluorescence microscope is an optical microscope that uses fluorescence and phosphorescence instead of, or in addition to, reflection and absorption, to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter.

In some embodiments, the plurality of training histological images, or tiles thereof, and/or input histological images, or tiles thereof, are captured digitally. In some embodiments, digital pathology slide scanners are used to capture images of biological samples. In some aspects, the digital pathology slide scanners can capture bright-field or fluorescent images. In some aspects, the digital pathology slide scanners have automated stages to automate imaging of a whole slide. The digital pathology slide scanners may also allow for Z stack images and/or live viewing of the biological sample. In some embodiments, the digital pathology slide scanners may hold around 1, 5, 10, 20, 30, 40, 50, 60, 70, 80, 90, 100 or more individual slides of biological samples. In some aspects, the digital pathology slide scanners may hold around 100, 150, 200, 250, 300, 350, 400, 450, 500, or more individual slides of biological samples. In some aspects, the digital histological images (e.g., digital histological training images, digital histological input images, or tiles thereof) are automatically uploaded to a computer or mobile device.

In some embodiments, histological images (e.g., training histological images, or tiles thereof, and/or input histological images, or tiles thereof) of the biological sample are captured with magnification. In some embodiments, the histological images are captured at about 2.5 x, 3 x, 4 x, 5 x, 6 x, 7 x, 8 x, 9 x, 10 x, 11 x, 12 x, 13 x, 14 x, 15 x, 16 x, 17 x, 18 x, 19 x, 20 x, or more magnification. In some embodiments, the magnification may be about 2.5 x or greater, 5 x or greater, 10 x or greater, or 15 x or greater. In some embodiments, the magnification may be about 20 x or less, 15 x or less, 10 x or less, 5 x or less, 2.5 x or less. In some embodiments, the image magnification may be between about 2.5 x and about 20 x, such as between any of about 2.5 x to about 5 x, about 5 x to about 10 x, or about 10 x to about 20 x. In some embodiments, the magnification varies across images tiles of a segmented whole slide histological image.

The magnification of the plurality of training histological images, or tiles thereof, and/or input histological images, or tiles thereof, may be achieved through eyepiece lenses, objective lenses, or a combination of thereof.

In some aspects, histological images (e.g., training histological images, or tiles thereof, and/or input histological images, or tiles thereof) are captured at high resolutions. In some embodiments, the high resolution capture of the histological images allows for detection of individual cells within a tissue. In some embodiments, the high resolution capture of the histological images allows for detection individual mitotic events (e.g., abnormal or normal mitotic events) within a cell within a tissue. In some embodiments, the resolution varies across a whole slide histological image. In some embodiments, the resolution varies across image tiles of a segmented whole slide histological image. High resolution histological images or histological image tiles may be captured at a resolution between about 256 x 256 pixels to about 10,000 x 10,000 pixels. In some embodiments, high resolution histological images or histological image tiles may be about 256 x 256 pixels, 300 x 300 pixels, 400 x 400 pixels, 500 x 500 pixels, 600 x 600 pixels, 700 x 700 pixels, 800 x 800 pixels, 900 x 900 pixels, 1,000 x 1,000 pixels, 2,000 x 2,000 pixels, 3,000 x 3,000 pixels, 4,000 x 4,000 pixels, 5,000 x 5,000 pixels, 6,000 x 6,000 pixels, 7,000 x 7,000 pixels, 8,000 x 8,000 pixels, 9,000 x 9,000 pixels, 10,000 x 10,000 pixels, or more. In some embodiments, the resolution of the histological image or histological image tile may include the resolutions and ranges above those described. In some embodiments, the resolution of the histological image or histological image tile may depend on the magnification of the histological image.

The disclosed methods may be utilized with histological images (e.g., training histological images, or tiles thereof, and/or input histological images, or tiles thereof), e.g., whole slide pathology (histological) images, of tissue samples that have been acquired using any of a variety of microscopy imaging techniques known to those of skill in the art. Examples include, but are not limited to, bright-field microscopy, dark-field microscopy, phase contrast microscopy, differential interference contrast (DIC) microscopy, fluorescence microscopy, confocal microscopy, confocal laser microscopy, super-resolution optical microscopy, scanning or transmission electron microscopy, and the like.

Any of a variety of image processing methods known to those of skill in the art may be used for image processing / pre-processing of the histological images (e.g., training histological images, or tiles thereof, and/or input histological images, or tiles thereof) described herein. Examples include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation methods (e.g., intensity thresholding, intensity clustering methods, intensity histogram-based methods, etc.), feature and pattern recognition methods (e.g., the generalized Hough transform for detecting arbitrary shapes, the circular Hough transform, etc.), and mathematical analysis methods (e.g., Fourier transform, fast Fourier transform, wavelet analysis, auto-correlation, etc.), or any combination thereof.

In some embodiments, the methods provided herein further comprise segmenting one or more of the input or training histological images into a plurality of histological image tiles prior to inputting the one or more input or training histological images into the machine-learning model. In some embodiments, the segmenting is implemented as part of the machine-learning model. In some embodiments, the whole histological images (e.g., whole training histological images and/or whole input histological images) are segmented into smaller image tiles. In some embodiments, the image tile is a segment of a whole histological image. In some embodiments, at least a portion of the plurality of training histological images is a tile of a whole training histological image. In some embodiments, at least a portion of the one or more whole input histological images is a tile of a whole input histological image.

In some embodiments, the image tiles are extracted from a whole histological image (e.g., whole training histological image and/or whole input histological image) using masking or other image processing techniques such as those described above. In some embodiments, a sliding window approach can be used to extract tiles from a whole histological image. In some aspects, about 5, 10, 15, 20, 25, 30, 40, 50, 60, 70, 80, 90, 100, 200, 400, 600, 800, 1,000, 1,500, 2,000, 2,500, 3,000, 3,500, 4,000, 4,500, 5,000, or more than 5,000 image tiles, may be extracted from each whole histological image of a plurality of whole histological images. In some instances, the number of image tiles (e.g., image patches) extracted from each whole histological image may have any value within the range of values described in this paragraph, e.g., 1,224 image tiles.

In some embodiments, the image tiles (e.g., images patches) extracted from a whole histological image (e.g., whole training histological image and/or whole input histological image) may be of different sizes. In some embodiments, the image tiles extracted from a histological image may all be of the same size. In some embodiments, the image tiles extracted from a whole histological image may be of a predetermined size. In some embodiments, the image tiles may all be of the same size when training a machine-learning model for a particular classification application, in order to ensure that the image tile patterns processed by the machine-learning model are consistent in terms of, e.g., field of view, and to ensure that the machine-learning model’s weights and the patterns learned are meaningful. In some instances, image tile size may be varied from experiment to experiment or from application to application. In some instances, image tile size can be considered a tunable parameter during the training method, such as any of the methods of training a machine-learning described herein.

In some embodiments, the image tile size (or a range of image tile sizes) is determined by the input histological image size expected to be characterized by the trained machine-learning model, or by other image size considerations. In some aspects, the size of the image tile may range from about 10 pixels to about 200 pixels. In some aspects, the size of the image tile may be at least 10 pixels, at least 50 pixels, at least 75 pixels, at least 100 pixels, at least 105 pixels, more. In some aspects, the size of the image tile may be at most 500 pixels, at most 200 pixels, at most 150 pixels, at most 120 pixels, at most 100 pixels, or at most 10 pixels. Any of the lower and upper values described in this paragraph may be combined to form a range included within the present disclosure, for example, in some instances the size of the image tile may range from about 100 pixels to about 105 pixels. Those of skill in the art will recognize that the size of the image tile may have any value within this range, e.g., about 2.8 x 103 pixels.

In some embodiments, the image tiles may be of a square or rectangular shape, e.g., 100 pixels x 100 pixels, or 1,000 pixels x 1,000 pixels. In some instances, the image tiles may be of irregular shape. In some embodiments, the histological images or the histological image tiles may be extracted from 1, 2, 3, 4, 5, 6, 7, 8, 9, 10 or more than 10 histological images of the same biological tissue.

### B. Training Data

The type of training data used for training a machine-learning model for use in the disclosed methods of characterizing a disease in a patient will depend on, for example, whether a supervised or unsupervised approach is taken, as well as on the objective to be achieved. In some instances, the training data may be continuously updated and used to update the machine-learning model(s) in a local or distributed network of one or more deployed pathology (histological) image analysis systems in real time. In some cases, the training data may be stored in a training database that resides on a local computer or server. In some cases, the training data may be stored in a training database that resides online or in a cloud.

In some embodiments, the machine-learning model is trained using a plurality of training histological images. In some embodiments, the training histological images comprise an image of a biological sample. In some embodiments, the biological sample comprises a tumor sample (e.g., a tumor sample or a portion thereof, obtained from a tumor slice of a patient). In some embodiments, the training histological images are stained images. In some embodiments, the training histological images are processed and captured according to any of the techniques described herein.

In some aspects, the training histological images may obtained from any source. In some embodiments, the machine-learning model is trained with histological images that are not publicly available. In some embodiments, the training histological images are obtained from a private source, such as an internal collaboration or licensed data. The training histological images may come from a clinic, a hospital system, a company or any other entity with access to histological images. In some embodiments, the training histological images are publicly available images. The training histological images may be downloaded from a publicly available database. In some aspects, the training histological images may be obtained from a publicly available database. The publicly available database may be a pan-cancer database. The publicly available databases may be available from, The Cancer Genome Atlas (TCGA) Pan-Cancer Atlas, Clinical Proteomic Tumor Analysis Consortium (CPTAC), National Lung Screening Trial, Osteosarcoma Pathology, Prostate Fused-MRI-Pathology, Prostate-MRI, Osteosarcoma Tumor Assessment, Lung Fused-CT-Pathology, AML-Cytomorphology LMU, Post-NAT-BRCA, SNL-Breast, C-NMC 2019, MiMM_SBILab, SN-AM, IvyGAP, or any other database with a histological image dataset.

In some embodiments, the machine-learning model is trained with histological images that are obtained from more than one source. In some embodiments, the machine-learning model is trained with histological images that come from 2, 3, 4, 5, 6, 7, 8, 9, 10 or more image datasets. In some embodiments, the machine-learning model is trained with a subset of histological images from one source.

In some embodiments, the machine-learning model is trained with about 1,000 histological images or image tiles thereof. In some embodiments, the machine-learning model is trained with about 100, 500, 1,000, 2,000, 3,000, 4,000, 5,000, 6,000, 7,000, 8,0000, 9,0000, 10,000, or more, histological images or image tiles thereof.

In some embodiments, the machine-learning model is trained with training histological images or tiles of training histological images captured from about 1,000 biological samples. In some embodiments, the biological samples are biological samples from the same patient. In some embodiments, at least a portion of the biological samples are biological samples from different patients. In some embodiments, the biological sample is a biological sample from a different patient. In some embodiments, the machine-learning model is trained with training histological images or training histological image tiles from about 100, 500, 1,000, 2,000, 3,000, 4,000, 5,000, 6,000, 7,000, 8,0000, 9,0000, 10,000, or more biological samples.

In some embodiments, the machine-learning model is trained with training histological images tiles of training histological images from about 1,000 patients. In some embodiments, the patients are different patients. In some embodiments, the machine-learning model is trained with training histological images or training histological image tiles from about 100, 500, 1,000, 2,000, 3,000, 4,000, 5,000, 6,000, 7,000, 8,0000, 9,0000, 10,000, or more patients.

In some embodiments, at least one training histological image from a plurality of training histological images or training histological tiles comes from the same biological sample. In some embodiments, more than one training histological image from a plurality of training histological images or training histological tiles comes from the same biological sample. In some embodiments, at least one training histological image from a plurality of training histological images or training histological tile comes from the same patient. In some embodiments, more than one training histological image from a plurality of training histological images or training histological tile comes from the same patient.

The training histological image (e.g., one or more training histological images or training histological image tiles thereof) may have a matched chromosomal instability pathological metric corresponding to the training histological image (e.g., the whole training histological image). In some aspects, each training histological image of a plurality of training histological images has a matched chromosomal instability pathological metric corresponding to the training histological image. In some embodiments, the corresponding chromosomal instability pathological metric is obtained for each training histological image of a plurality of training histological images. In some embodiments, the corresponding chromosomal instability pathological metric is computed for each training histological image of a plurality of training histological images. In some embodiments, the computing is implemented as a machine-learning model, such as any of the machine-learning models described herein.

A chromosomal instability pathological metric may be any metric that serves as a measure of the chromosomal instability status of the biological sample of the input histological image or images tiles thereof. In some embodiments, the chromosomal instability may be a numerical chromosomal event, wherein the events result in gains or losses of whole chromosomes. In some aspects, the chromosomal instability may be a structural chromosomal instability, wherein the events result in amplification, deletions, inversions, and translocations of chromosomal regions that range in size from single genes to whole chromosomal arms. In some embodiments, the chromosomal instability pathological metric is computed from DNA. In some embodiments, the chromosomal instability pathological metric is computed using a stained image (e.g., a stained training histological image of a biological sample). In some embodiments, the image is stained with hematoxylin and eosin (H&E) and/or 4′,6-diamidino-2-phenylindole (DAPI) stain.

In some preferred embodiments, the corresponding chromosomal instability pathological metric is computed from DNA from the biological sample of the training histological image or image tiles thereof. In some embodiments, the chromosomal pathological metric and the biological sample of the training histological image, or image tiles thereof, come from the same individual (e.g., a patient). In some embodiments, the DNA from which the corresponding chromosomal pathological metric is computed is from the same patient as the biological sample of the training histological image. In some embodiments, the corresponding chromosomal instability pathological metric is matched with a training histological image or image tiles thereof.

The DNA used to compute a corresponding chromosomal pathological metric for an input histological image, or images tiles thereof, may be obtained from a sample. In some embodiments, the sample is a biological sample. In some embodiments, the biological sample comprises a bodily fluid comprising a DNA sample, such as a sample comprising a blood sample, serum sample, convalescent plasma sample, oropharyngeal sample, including that obtained from an oropharyngeal swab, nasopharyngeal sample, including that obtained from a nasopharyngeal swab, buccal sample, bronchoalveolar lavage sample, including that obtained from an endotracheal aspirator, a urine sample, a sweat sample, a sputum sample, a salivary sample, a tear sample, a bodily excretion sample, or cerebrospinal fluid sample. In some embodiments, the biological sample comprises a solid, such as a sample comprising a fecal sample, a skin sample, a nail sample, or plucked and shed hairs. In some embodiments, the biological sample comprises tumor DNA. In some embodiments, the biological ample is a tumor DNA sample.

In some embodiments, the DNA sample is from an individual (e.g., a patient). In some embodiments, the individual is a mammal, such as a human, bovine, horse, feline, canine, rodent, or primate. In some embodiments, the machine-learning method described herein comprises obtaining the sample from an individual (e.g., a patient). In some embodiments, the individual is the same individual as from which the biological sample of the training histological image, or image tiles thereof, was obtained. In some embodiments, the DNA sample is collected form an individual using a check swab, nasopharyngeal swab, oropharyngeal swab, standard blood collection needle, fine needle aspiration, vacutainer tubes, ear punches, or any other suitable method. In some embodiments, the DNA sample is collected from a tumor in an individual. In some embodiments, the DNA sample has been preserved prior to use in a method described herein. In some embodiments, the DNA sample was previously frozen.

In some embodiments, the sample (e.g., the DNA sample) is suspected of containing, and in some instances contains, an occurrence of chromosomal instability. In some embodiments, the sample is taken from an individual suspected of having, and in some instances has, a disease. In some embodiments, the sample is taken from an individual suspected of having cancer. Thus, in some embodiments, the sample contains tumor DNA. In some embodiments, the occurrence of chromosomal instability may be used to compute a chromosomal instability pathological metric associated with a disease of an individual, such as a human disease (e.g., cancer). Thus, the sample may be used to determine a chromosomal instability pathological metric corresponding to a training histological image (e.g., a matched training histological image), or image tiles thereof.

In some embodiments, the chromosomal instability pathological metric is used to predict the pathological status of the training histological images, or image tiles thereof. In some embodiments, the pathological status is described as a metric, wherein the pathological status metric may include of a probability of high chromosomal instability in the image, a continuous chromosomal instability score, a binary classification of high or low chromosomal instability, or a combination thereof. Exemplary chromosomal instability pathological metrics include, but are not limited to bulk genomic aneuploidy burden, fraction of the genome altered (FGA), cyclic GMP-AMP synthase (cGAS)-stimulator of interferon genes (STING) activity, CIN23 gene signature, increased copy number heterogeneity, gain/loss of chromosomes, type I interferon expression, increased presence of micronuclei, etc. The chromosomal instability pathological metric can be evaluated using any suitable technique known in the art, wherein the technique is related to the specific metric to be determined. For example, the chromosomal instability pathological metric can be evaluated by DNA sequencing (e.g., whole genome sequencing), RNAseq, qPCR, single-sample gene set enrichment analysis (ssGSEA), fluorescent labeling of chromatin-associated proteins, modified gene editing systems, DNA staining (e.g., DAPI staining), Giemsa banding and inverted DAPI counterstaining, fluorescence in situ hybridization (FISH), spectral karyotyping and multiplex-banding, quantitative imaging microscopy, imaging flow cytometry, sc-CGH sc-sequencing, or any combination thereof.

FGA, as used herein, refers to the ratio of sum of altered genome size/total genome size analyzed (e.g., percentage of the genome that has been affected by copy number gains or losses). In some embodiments, the FGA is calculated using whole genome sequencing data. In some embodiments, the whole genome sequencing data is publicly available from a database, such as any of the databases described herein. In some embodiments, the database is The Cancer Genome Atlas (TCGA) Program.

In some embodiments, the FGA is calculated using exome sequencing based copy number variation (CNV) data. In some embodiments, the whole exome sequencing based copy number variation (CNV) data is publicly available from a database, such as any of the databases described herein. In some embodiments, copy number segments are downloaded from a publicly available from a database, such as any of the databases described herein. In some embodiments, the database is The Cancer Genome Atlas (TCGA) Program. In some embodiments, copy number segments with log transformed mean copy number values larger than 0.2 or less than -0.2 are treated as altered segments, respectively. For additional description of FGA, see, for example, Salas et al., Epigenetics (2017) vol. 12(7): 561-574 and Ali Hassan et al., PLoS One (2014) vol. 9(4):e92553, which are herein incorporated by reference in their entirety.

In some embodiments, the FGA is used as the corresponding chromosomal instability pathological metric matched to a training histological image. In some embodiments, the degree of FGA (e.g., computed using log transformed mean copy number values) is used to predict the pathological status of the training histological images. In some embodiments, the pathological status is described as a metric, wherein the pathological status metric may include a probability of high chromosomal instability in the image, a continuous chromosomal instability score, a binary classification of high or low chromosomal instability, or a combination thereof. The FGA may be used to predict the pathological status of the training histological image as “low chromosomal instability” when the sample comprises copy number segments with log transformed mean copy number values less than about 0.3, such as less than any of about 0.25, 0.2, 0.15, 0.1 or less. The FGA may be used to predict the pathological status of the training histological image as “high chromosomal instability” when the sample comprises copy number segments with log transformed mean copy number values greater than about 0.3, such as greater than any of about 0.35, 0.4, 0.45, 0.5 or greater.

In some embodiments, a disease subtype is used as the corresponding chromosomal instability pathological metric matched to a training histological image. In some embodiments, a cancer subtype is used as the corresponding chromosomal instability pathological metric matched to a training histological image. In some embodiments, the disease or cancer subtype has a unique chromosomal instability metric compared to a different disease or cancer subtype.

Staining of a training histological image, or image tiles thereof, may be performed to evaluate a corresponding chromosomal instability pathological metric matched to a training histological image, or image tiles thereof. In some embodiments, the training histological image, or images tiles thereof, itself is stained to evaluate the corresponding chromosomal instability pathological metric. In some embodiments, a secondary histological image (e.g., a histological image that is not the training histological image) is stained to evaluate the corresponding chromosomal instability pathological metric. The secondary histological image may be from the same biological sample as the biological sample from the training histological image. In some embodiments, the secondary histological image is from a biological sample of the same individual (e.g., the same patient) as the biological sample from the training histological image.

The stain may be any suitable stain known in the art that may be used to evaluate the corresponding chromosomal instability pathological metric of interest. In some embodiments, the stain is a DNA stain, such as DAPI. In some embodiments, the stain is a histological stain to identify tissues, cellular structures, and/or the distribution of chemical substances, such as a H&E stain.

In some embodiments, a histological image (e.g., a training histological image) is stained to evaluate the presence of micronuclei. Micronuclei may serve as a corresponding chromosomal instability pathological metric matched to a training histological image. In some aspects, the presence of micronuclei is a hallmark of chromosome instability. In some embodiments, the micronuclei of a histological image are stained with DAPI to serve as the corresponding chromosomal instability pathological metric matched to a training histological image. In some embodiments, the degree of micronuclei present in a histological image is used to predict the pathological status of the training histological images. In some embodiments, the pathological status is described as a metric, wherein the pathological status metric may include a probability of high chromosomal instability in the image, a continuous chromosomal instability score, a binary classification of high or low chromosomal instability, or a combination thereof. In some embodiments, the histological images, or image tiles thereof, may comprise greater than about 5, 10, 15, 20, 25, 30, 35, 40, 45, 50, 55, 60, 65, 70, 75, 80, 85, 90, 95, 100, 200, 300, 400, 500, 1,000, or more, micronuclei. In some embodiments, the histological images, or image tiles thereof, may contain a cell that comprises greater than about 5, 10, 15, 20, 25, 30, 35, 40, 45, 50, 55, 60, 65, 70, 75, 80, 85, 90, 95, 100, 200, 300, 400, 500, 1,000, or more, micronuclei.

In some embodiments, the training histological images, or image tiles thereof, differ in the level of the chromosomal instability pathology metric. In some embodiments, the training histological images, or image tiles thereof, may differ in the level of the chromosomal instability pathology metric by about 0, 5, 10, 15, 20, 25, 30, 35, 40, 45, 50, 55, 60, 65, 70, 75, 80, 85, 90, 95, or 100%. In some embodiments, the level of the chromosomal instability pathology metric may be an aggregation of the chromosomal instability pathology metric identified in the input histological image tiles of one input histological image.

In some embodiments, the chromosomal instability pathology metric may be based on a numerical chromosomal instability, wherein the pathology results in gains or losses of whole chromosomes. In some aspects, the chromosomal instability pathology metric may be based on a structural chromosomal instability, wherein the pathology results in amplification, deletions, inversions, and/or translocations of chromosomal regions, that range in size from single genes to whole chromosomal arms.

### C. Machine-Learning Models

In some embodiments, the disclosed methods include use one or more machine-learning methods to pre-process and/or analyze histological images (e.g., training histological images, or tiles thereof, and/or input histological images, or tiles thereof). A variety of machine-learning models may be implemented for the disclosed methods. The models may comprise a unsupervised machine-learning model, a weakly-supervised machine-learning model, a human-in-the-loop machine-learning model, a supervised machine-learning model, a deep learning machine-learning model, etc., or any combination thereof.

In some embodiments, unsupervised machine-learning models are used to draw inferences from training histological images and/or training histological image tiles when they are not paired with a chromosomal instability pathological metric, in accordance with the methods provided herein. One example of a commonly used unsupervised machine-learning model is cluster analysis. Cluster analysis may be used for exploratory data analysis to find hidden patterns or groupings in multi-dimensional data sets. Other examples of unsupervised machine-learning models include, but are not limited to, artificial neural networks, association rule machine-learning models, etc.

In some aspects, a weakly-supervised machine-learning model is used in accordance with the methods provided herein. In some aspects, these weakly-supervised machine-learning models may use both training histological images, or image tiles thereof, with a matched chromosomal instability pathological metric and/or training histological images, or image tiles thereof, without a matched chromosomal instability pathological metric

In some embodiments, a human-in-the-loop machine-learning model is used in accordance with the methods provided herein. In human-in-the-loop machine-learning models may include initial iterations of model training that are unsupervised, followed by human interactions to add or modify the computer labels (e.g., chromosomal instability pathological metric labels). In some embodiments, humans may add information regarding a chromosomal instability pathological metric to the machine-learning model after the machine-learning model has started to learn from the training histological images and/or training histological image tiles.

In some embodiments, a supervised machine-learning model is used in accordance with the methods provided herein. Supervised machine-learning models may rely on training histological images, or image tiles thereof, and a matched chromosomal instability pathological metric. The machine-learning models may infer the relationships between a set of training histological images and/or training histological image tiles, and user specified matched chromosomal instability pathological metric for the training histological images and/or training histological image tiles. The training data is used to “teach” the supervised machine-learning model and comprises a set of paired training examples. Examples of supervised machine-learning models include but are not limited to support vector machines (SVMs), artificial neural networks (ANNs), etc.

In some embodiments, Automated Machine learning (AutoML) can be used to automate the machine-learning model development. In some embodiments, AutoMI, can be used to automate training and tuning the machine-learning model. In some embodiments, AutoMI, can be used for a classification machine-learning model. In some embodiments, AutoMI, can be used for a regression machine-learning model.

In some embodiments, artificial neural network models (ANNs) are used in accordance with the methods provided herein. In the context of the present disclosure, ANNs are machine-learning models inspired by the structure and function of the human brain. ANNs comprise an interconnected group of nodes organized into multiple layers. The ANN architecture may comprise at least an input layer, one or many hidden layers, and an output layer. In some embodiments, the ANN may have about 1, 2, 3, 4, 5, 6, 7, 8, 9, 10, or more hidden layers. Deep learning models are large ANN comprising many layers of coupled “nodes” between the input layer and output layer that may be used, for example, to map histological images or histological image tiles to chromosomal instability pathological metrics and corresponding pathological status of input histological images related to chromosomal instability.

The ANN may comprise any total number of layers, and any number of hidden layers, where the hidden layers function as trainable feature extractors that allow mapping of a set of input histological images or input histological image tiles to a preferred output value or set of output values such as identified mitotic events or chromosome instability scores. Each layer of the neural network comprises a number of nodes (or “neurons”). A node receives input that comes either directly from the input data (e.g., histological images, histological image tiles, or matched chromosomal instability pathological metrics) or from the output of nodes in previous layers, and performs a specific operation, e.g., a summation operation.

In some cases, a connection from an input to a node is associated with a weight (or weighting factor). In some cases, the node may, for example, sum up the products of all pairs of inputs, Xi, and their associated weights, Wi. In some cases, the weighted sum is offset with a bias, b. In some cases, the output of a neuron may be gated using a threshold or activation function, f, which may be a linear or non-linear function. In some embodiments, the activation function may be, for example, a rectified linear unit (ReLU) activation function or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training histological images. For example, the parameters may be trained using the input data from a training histological image or training histological image tiles a gradient descent or backward propagation method so that the chromosomal instability pathological metric and/or pathological status related to chromosomal instability computed by the ANN are consistent with the training histological image or training histological image tile examples. The adjustable parameters of the model may be obtained using, e.g., a back propagation neural network training process that may or may not be performed using the same hardware as that used for processing histological images and/or performing biological sample preparation.

Other specific types of deep machine-learning models, e.g., convolutional neural networks (CNNs) (often used for the processing of image data from machine vision systems) may also be used in implementing the disclosed methods. CNNs are commonly composed of layers of different types: convolution, pooling, upscaling, and fully-connected node layers. In some cases, an activation function such as rectified linear unit may be used in some of the layers. In a CNN architecture, there can be one or more layers for each type of operation performed. A CNN architecture may comprise any number of layers in total, and any number of layers for the different types of operations performed. The simplest convolutional neural network architecture starts with an input layer followed by a sequence of convolutional layers and pooling layers, and ends with fully-connected layers. Each convolution layer may comprise a plurality of parameters used for performing the convolution operations. Each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parameteric rectified linear unit, exponential linear unit, softPlus, bent identity, softExponential, Sinusoid, Sine, Gaussian, the sigmoid function and various others. The convolutional, pooling and ReLU layers may function as learnable features extractors, while the fully connected layers may function as a machine-learning classifier. As with other artificial neural networks, the convolutional layers and fully- connected layers of CNN architectures typically include various adjustable computational parameters, e.g., weights, bias values, and threshold values, that are trained in a training phase as described above.

For any of the various types of ANN models that may be used in the methods disclosed herein, the number of nodes used in the input layer of the ANN (which enable input of data from, for example, histological images, histological image tiles, a multi-dimensional image feature data set, and/or other types of input data) may range from about 10 to about 20,000 nodes. In some instances, the number of nodes used in the input layer may be at least 10, at least 50, at least 100, at least 200, at least 300, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 12,000, at least 14,000, at least 16,000, at least 18,000, or at least 20,000. In some instances, the number of node used in the input layer may be at most 20,000, at most 18,000, at most 16,000, at most 14,000, at most 12,000, at most 10,000, at most 9,000, at most 8,000, at most 7,000, at most 6,000, at most 5,000, at most 4,000, at most 3,000, at most 2,000, at most 1,000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes. In some instances, the number of nodes used in the input layer may be a tunable parameter of the ANN model.

In some instances, the total number of layers used in the ANN models used to implement the disclosed methods (including input and output layers) may range from about 3 to about 1,000, or more. In some instances the total number of layers may be at least 3, at least 4, at least 5, at least 10, at least 15, at least 20, at least 40, at least 60, at least 80, at least 100, at least 200, at least 300, at least 400, at least 500, at least 600, at least 700, at least 800, at least 900, or at least 1,000. In some instances, the total number of layers may be at most 1000, at most 800, at most 600, at most 400, at most 200, at most 100, at most 80, at most 60, at most 40, at most 20, at most 15, at most 10, at most 5, at most 4, or at most 3. Those of skill in the art will recognize that, in some cases, the total number of layers used in the ANN model may have any value within this range, for example, 8 layers.

In some instances, the total number of learnable or trainable parameters, e.g., weighting factors, biases, or threshold values, used in the ANN may range from about 10 to about 10,000,000. In some instances, the total number of learnable parameters may be at least 10, at least 100, at least 500, at least 1,000, at least 2,000, at least 3,000, at least 4,000, at least 5,000, at least 6,000, at least 7,000, at least 8,000, at least 9,000, at least 10,000, at least 20,000, at least 40,000, at least 60,000, at least 80,000, at least 100,000, at least 250,000, at least 500,000, at least 750,00, at least 1,000,000, at least 2,500,000, at least 5,000,000, at least 7,500,000, or at least 10,000,000. Alternatively, the total number of learnable parameters may be any number less than 100, any number between 100 and 10,000, or a number greater than 10,000. In some instances, the total number of learnable parameters may be at most 10,000,000, at most 7,500,000, at most 5,000,000, at most 2,500,000, at most 1,000,000, at most 750,000, at most 500,000, at most 250,000, at most 100,000, at most 80,000, at most 60,000, at most 40,000, at most 20,000, at most 10,000, at most 9,000, at most 8,000, at most 7,000, at most 6,000, at most 5,000, at most 4,000, at most 3,000, at most 2,000, at most 1,000, at most 500, at most 100, or at most 10. Those of skill in the art will recognize that the total number of learnable parameters used may have any value within this range, for example, about 2,200 parameters.

In some embodiments, implementation of the disclosed methods may comprise use of an autoencoder model. Autoencoders (also sometimes referred to as an auto-associator or Diabolo networks) are artificial neural networks used for unsupervised, efficient mapping of input data, e.g., training histological images, to an output value, e.g., an image cluster and/or chromosomal instability classifications. Autoencoders are often used for the purpose of dimensionality reduction, i.e., the process of reducing the number of random variables under consideration by deducing a set of principal component variables.

Dimensionality reduction may be performed, for example, for the purpose of feature selection (e.g., selection of the most relevant subset of the image features presented in the original training histological image data set comprising training histological images and/or training histological image tiles) or feature extraction (e.g., transformation of image feature data in the original, multi-dimensional image space to a space of fewer dimensions as defined, e.g., by a series of feature parameters).

Any of a variety of different autoencoder models known to those of skill in the art may be used in the disclosed methods. Examples include, but are not limited to, stacked autoencoders, denoising autoencoders, variational autoencoders, or any combination thereof. Stacked autoencoders are neural networks consisting of multiple layers of sparse autoencoders in which the output of each layer is wired to the input of the successive layer. Variational autoencoders (VAEs) are autoencoder models that use the basic autoencoder architecture, but that make strong assumptions regarding the distribution of latent variables. They use a variational approach for latent representation learning, which results in an additional loss component, and may require the use of a specific training method called Stochastic Gradient Variational Bayes (SGVB).

In some embodiments, implementation of the disclosed methods and systems may comprise the use of a deep convolutional generative adversarial network (DCGAN). DCGANs are a class of convolutional neural networks (CNNs) used for unsupervised learning that further comprise a generative adversarial network (GANs), i.e., they comprise a class of models implemented by a system of two neural networks contesting with each other in a zero-sum game framework. One network generates candidate images (or solutions) and the other network evaluates them. Typically, the generative network learns to map from a latent space (i.e., a representation of compressed data in which similar data points are closer together in space; latent space is useful for learning data features and for finding simpler representations of data for analysis) to a particular data distribution of interest, while the discriminative network discriminates between instances from the true data distribution and the candidate images (or solutions) produced by the generator. The generative network’s training objective is to increase the error rate of the discriminative network (i.e., to “fool” the discriminator network) by producing novel synthesized instances that appear to have come from the true data distribution). In practice, a known dataset serves as the initial training data for the discriminator. Training the discriminator involves presenting it with samples from the dataset, until it reaches some level of accuracy. Typically the generator is seeded with a randomized input that is sampled from a predefined latent space (e.g., a multivariate normal distribution). Thereafter, samples synthesized by the generator are evaluated by the discriminator. Backpropagation is applied in both networks so that the generator produces better images, while the discriminator becomes more skilled at flagging synthetic images. The generator is typically a deconvolutional neural network, and the discriminator is a convolutional neural network. In some instances, implementation of the disclosed methods and systems may comprise the use of a Wasserstein generative adversarial network (WGAN), a variation of the DCGAN structure that uses a slightly modified architecture and/or a modified loss function.

In some embodiments, the disclosed methods may use a cluster method to cluster histological images or histological image tiles according to features. Any variety of clustering methods known to those skilled in the art may be used. Clustering methods may include, but are not limited to, k-means clustering methods, hierarchical clustering methods, mean-shift clustering methods, density-based spatial clustering methods, expectation-maximization clustering methods, and mixture model (e.g., mixtures of Gaussians) clustering methods.

In some embodiments, hierarchical clustering method may be used to group histological images or histological image tiles into groups or clusters. In some embodiments, a feature space (e.g., a chromosomal instability pathological metric) in a histological image or histological image tiles is clustered. Hierarchical clustering methods may be used to identify a set of clusters such as histological images with similar normal or abnormal mitotic events. Initially, each data point is treated as a separate cluster. A distance matrix for pairs of data points is calculated, and the method then repeats the steps of: (i) identifying the two clusters that are closest together, and (ii) merging the two most similar clusters. The iterative process continues until all similar clusters have been merged.

In some embodiments, a clustering model may use a Gaussian mixture model. Gaussian mixture models are probabilistic models that assume all data points (e.g., histological image or histological image segment) in a data set may be represented by a mixture of a finite number of Gaussian distributions with unknown peak height, position, or standard deviations. The approach is similar to generalizing a k-means clustering method to incorporate information about the covariance structure of the data as well as the centers of the latent Gaussians.

Any of a variety of commercial or open-source program packages, program languages, or platforms known to those of skill in the art may be used to implement the machine-learning models of the disclosed methods and systems. Examples include, but are not limited to, Shogun (www.shogun-toolbox.org), Mlpack (www.mlpack.rog), R (r-project.org), Weka (www.cs.waikato.ac.nz/ml/weka/), Python (www.python.org), and/or Matlab (MathWorks, Natick, MA).

### D. Statistical Data Analysis

In some embodiments, the disclosed methods and systems may comprise use of statistical data analysis techniques. In some embodiments, statistical data analysis techniques include processes for histological image processing and/or methods incorporated into the machine-learning model to identify key components that underlie observed variation in pathological status metrics related to chromosomal instability. The combination of one or more statistical analysis models, e.g. principal component analysis (PCA), may be used in combination with a machine-learning model, to identify key components (or attributes) that comprise heterogeneity of pathological status metrics related to chromosomal instability events in histological images. In some embodiments, the basis set of key attributes identified by a statistical and/or machine-learning-based analysis may comprise 1 key attribute, 2 key attributes, 3 key attributes, 4 key attributes, 5 key attributes, 6 key attributes, 7 key attributes, 8 key attributes, 9 key attributes, 10 key attributes, 15 key attributes, 20 key attributes, or more.

Any of a variety of suitable statistical analysis methods known to those of skill in the art may be used in performing the disclosed methods. Examples include, but are not limited to, principal component and other eigenvector-based analysis methods, regression analysis, probabilistic graphical models, or any combination thereof. In some embodiments, the statistical analysis method is a probability metric for a predicted classification of a corresponding chromosomal instability pathological metric matched to a histological training image. In some embodiments, the statistical analysis method is a probability metric for a predicted pathological status (e.g., a pathological status metric) of an input histological image. In some embodiments, the pathological status metric may include a probability of high chromosomal instability in the image, a continuous chromosomal instability score, a binary classification of high or low chromosomal instability, or a combination thereof. A threshold value may be applied to these metrics, for example, a confidence threshold that can be calculated based on, for instance, the total genome size analyzed.

### E. Machine-Learning Model Output

The machine-learning model may output a predicted pathological status (e.g., pathology predicted chromosomal instability score) for the input histological image or input histological image tiles. In some embodiments, the machine-learning model uses the matched chromosomal instability pathological metric and the training histological images to predict the pathological status of the input histological image or input histological image tiles. In some embodiments, the pathological status relates to the chromosomal instability pathological metric in the input histological image or input histological image tiles. The pathological status may be described as a pathological status metric, wherein the pathological status metric is related to a chromosomal instability of the biological sample of the input histological image or input histological image tiles. The pathological status metric (e.g., pathology predicted chromosomal instability score) of a particular input histological image may be used to characterize a disease in a patient, such as by any of the methods for characterizing a disease provided herein.

In some embodiments, the machine-learning model output does not require human intervention. Thus, the machine-learning model described herein may be fully automated. In some embodiments, the machine-learning model uses transfer learning and feature aggregation to accurately discriminate a pathological status metric, such as chromosomal instability, of histopathology slides (e.g., input histological images or input histological image tiles) without human intervention. In some embodiments, the predicted pathological status metric is a probability of high chromosomal instability of the biological sample in the input histological image or input histological image tiles, a continuous chromosomal instability score of the biological sample in the input histological image or input histological image tiles, a binary classification of high or low chromosomal instability of the biological sample in the input histological image or input histological image tiles, or any combination thereof.

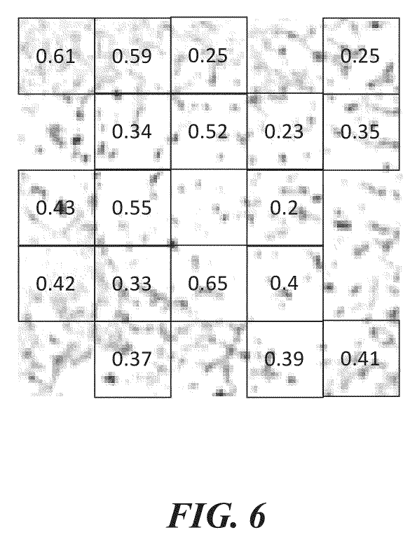

An exemplary output of a machine-learning model of the present disclosure is shown in FIG. 6. The predicted pathological status metric may be displayed to a user. In some embodiments, the predicted pathological status metric is displayed as an output of the machine-learning model described herein. In some embodiments, the predicted pathological status metric is displayed as text on the input histological images or input histological image tiles (see, e.g., FIG. 6). In some embodiments, the predicted pathological status metric is a patch-level prediction, wherein the predicted pathological status metric is computed on each input histological image tile. Patch-level predicted pathological status metrics may reveal intra-image heterogeneity (e.g., intra-tumor heterogeneity of chromosomal instability). Thus, in some embodiments, the biological sample of the input histological image or input histological image tiles comprise different patch-level predicted pathological status metrics. The patch-level predicted pathological status metrics may be averaged to determine the predicted pathological status metric of the biological sample of a whole input histological image. In some embodiments, the biological sample of the whole input histological image is predicted to be “high chromosomal instability” status or “low chromosomal instability” status using the predicted pathological status metric. In some embodiments, the chromosomal instability status of the biological sample of the input histological images or input histological image tiles is used to characterize a disease (e.g., cancer). For example, a biological sample predicted to have high chromosomal instability status may be more likely to have advanced disease (e.g., cancer) pathology compared to a biological sample predicted to have low chromosomal instability.

The predicted pathological status metric may be the probability of high chromosomal instability in the biological sample of the input histological image or input histological image tiles. In some embodiments, the probability of high chromosomal instability is determined in the biological sample of the input histological image or input histological image tiles using a machine-learning model trained using a plurality of training histological images and one or more matched chromosomal instability pathological metrics corresponding to the plurality of training histological images. The machine-learning model may assign a probability to the high chromosomal instability of biological sample of the input histological image or input histological image tiles for use in the model output. A prediction of the probability of high chromosomal instability in the biological sample of the input histological image or input histological image tiles may be based on a chromosomal instability pathological metric and matched training histological images used to train the machine-learning model. In some embodiments, the chromosomal instability pathological metric is any of the chromosomal instability pathological metrics described herein, such as bulk genomic aneuploidy burden (e.g., fraction of the genome altered, “FGA”).

In some aspects, the predicted pathological status metric is a continuous chromosomal instability score. The continuous chromosomal instability score is determined in the biological sample of the input histological image or input histological image tiles using a machine-learning model trained using a plurality of training histological images and one or more matched chromosomal instability pathological metrics corresponding to the plurality of training histological images. In some embodiments, the continuous chromosomal instability score captures ongoing chromosomal instability by evaluating biological sample of cells that are activity dividing. The machine-learning model may assign a continuous chromosomal instability score to the biological sample of the inputted histological image or inputted histological image segments for use in the model output. A prediction of the continuous high chromosomal instability in the biological sample of the input histological image or input histological image tiles may be based on a chromosomal instability pathological metric and matched training histological images used to train the machine-learning model. In some embodiments, the chromosomal instability pathological metric is any of the chromosomal instability pathological metrics described herein that capture ongoing chromosomal instability, such as the presence of micronuclei.

In some aspects, the predicted pathological status metric is a binary classification of high or low chromosomal instability. The binary classification of high or low chromosomal instability is determined in the biological sample of the inputted histological image or inputted histological image segments using a machine-learning model trained using a plurality of training histological images and one or more matched chromosomal instability pathological metrics corresponding to the plurality of training histological images. In some embodiments, the binary classification of high or low chromosomal instability is based on a specified threshold value of chromosomal instability. In some embodiments, the binary classification is high or low genomic chromosomal instability (e.g., chromosomal instability correlated with fraction of the genome altered). In some embodiments, the binary classification is high or low predicted pathological status metric of chromosomal instability (e.g., predicted pathological status of an inputted histological image or inputted histological image segment). The machine-learning model may assign a binary classification of high or low chromosomal instability to the biological sample of the inputted histological image or inputted histological image segments for use in the model output. A prediction of the binary classification of high or low chromosomal instability in the biological sample of the input histological image or input histological image tiles may be based on a chromosomal instability pathological metric and matched training histological images used to train the machine-learning model. In some embodiments, the chromosomal instability pathological metric is any of the chromosomal instability pathological metrics described herein, such as bulk genomic aneuploidy burden (e.g., fraction of the genome altered, “FGA”).

### F. Processors, Computer Systems, and Distributed Computer Environments

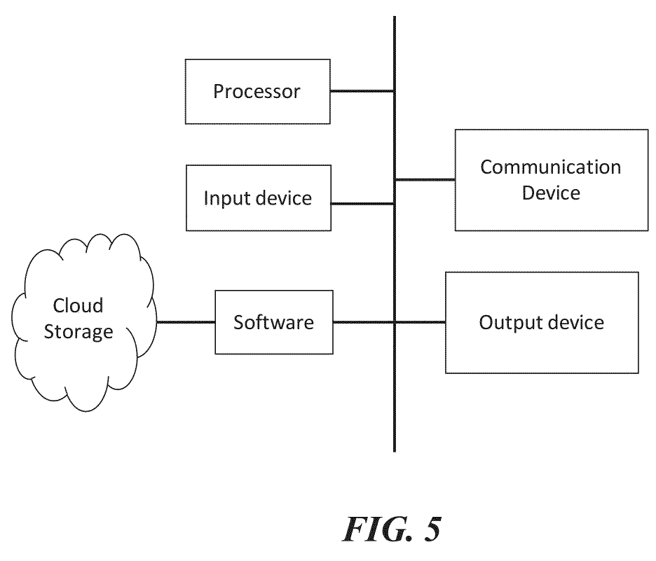

In some embodiments, one or more processors may be used to implement the machine-learning-based methods and systems disclosed herein. In addition to running the machine-learning and/or statistical analysis methods used to implement the disclosed methods, the one or more processors may be used for inputting data (e.g., histological images, segments of histological images), to the machine-learning model, or for outputting a result from the machine-learning model. The one or more processors may comprise a hardware processor such as a central processing unit (CPU), a graphic processing unit (GPU), a general-purpose processing unit, or other computing platform such as a cloud based computing platform. An exemplary cloud based computing platform according to some embodiments is illustrated in FIG. 5. The processor may be comprised of any of a variety of suitable integrated circuits, microprocessors, logic devices, field programmable gate arrays (FPGAs), and the like. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations.

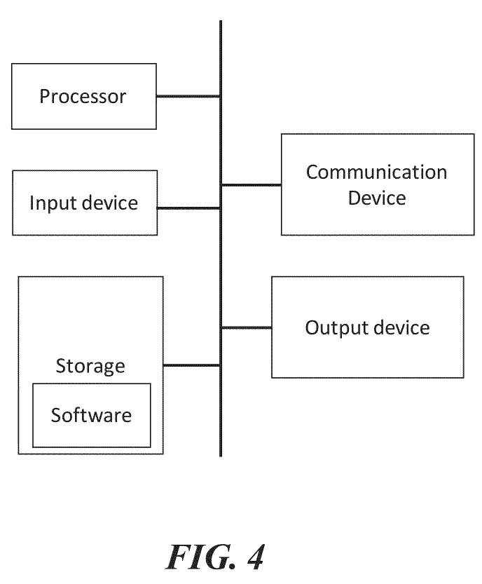

FIG. 4 illustrates an example computer system in accordance with one or more examples described herein. As shown in FIG. 4, a computer system may comprise one or more processors, an input device, an output devices, storage, and a communication device. The computer system may be any suitable type of microprocessor-based device, such as a personal computer, workstation, server, or handheld device. The computer components described herein may be connectable or integrated into the computer.

In some embodiments, the input device may be any suitable device that provides input, such as a touch screen, keyboard or keypad, mouse, or voice-recognition device. In some aspects, the output device can be any suitable device that provides output, such as a touch screen, haptics device, or speaker. In some embodiments, the storage can be any suitable device that provides storage, such as an electrical, magnetic, or optical memory including RAM, cache, hard drive, or removable storage disk. In some embodiments, storage can be in the form of an external computing cloud. The communication device may be any suitable device capable of transmitting and receiving signals over a network, such as a network interface chip or device.

In some embodiments, a computer-readable storage medium may store programs with the instructions for one or more processor of an electronic device to run the machine-learning model disclosed herein. The computer-readable storage medium disclosed herein can be stored in memory or storage and can be executed by a processor. The computer-readable storage medium can be stored or propagated for use by or in connection with an instruction execution system, apparatus, or device, such as those described above, that can fetch instructions associated with the program from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

The computer system described herein may be connected to a network, which can be any suitable type of interconnected communication system. The network can implement any suitable communication protocol and can be secured by any suitable security protocol. The network may comprise network links of any suitable arrangement that can implement the transmission and reception of network signals, such as wireless network connections, T1 or T3 lines, cable networks, DSL, or telephone lines.

The computer system described herein may implement any operating system for operating on the network. The computer-readable storage medium can be written in any suitable programming language, such as C, C++, Java, or Python. In some embodiments, programs embodying the functionality of the present disclosure can be deployed in different configurations, such as in a client/server arrangement or through a web browser as a web-based application or web service, for example.

In some embodiments, the methods and systems of the present disclosure may be implemented through 1, 2, 3, 4, 5, or more, machine-learning models. A machine-learning model can be implemented by way of a coded program instructions upon execution by the central processing unit.

### IV. System

One aspect of the present application provides a system for using the methods described herein to train a machine-learning model using a plurality of training histological images and one or more matched chromosomal instability pathological metrics corresponding to the plurality of training histological images. In another aspect, provided herein is a system for characterizing a disease in a patient with machine-learning, comprising: one or more processors; a memory; and one or more programs with instructions for: receiving data representing one or more input histological images of a biological sample; and, classifying a pathological status of the biological sample in the one or more input histological images using a trained machine-learning model trained using a plurality of training histological images and one or more matched chromosomal instability pathological metrics corresponding to the plurality of training histological images. In some embodiments, the system for characterizing a disease in a patient with machine-learning outputs a pathological status of the biological sample in the one or more inputted histological images using the trained machine-learning model.

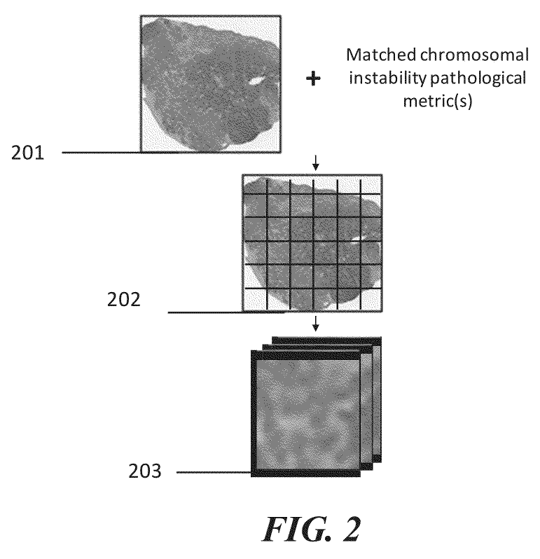

FIG. 2 illustrates an exemplary schematic of a method for training a machine-learning model completed by the machine-learning model systems described herein. A training histological image of a solid tumor is obtained, 201. In some embodiments, a stained training histological image is obtained (e.g., a hematoxylin and eosin (H&E) stained image or a 4′,6-diamidino-2-phenylindole (DAPI) stained image). One or more matched chromosomal instability pathological metrics corresponding to the training histological image are additionally obtained, 201. Exemplary chromosomal instability pathological metrics include, but are not limited to bulk genomic aneuploidy burden, fraction of the genome altered (FGA), cyclic GMP-AMP synthase (cGAS)-stimulator of interferon genes (STING) activity, increased copy number heterogeneity, CIN23 gene signature, gain/loss of chromosomes, type I interferon expression, increased presence of micronuclei, etc. In some preferred embodiments, the corresponding chromosomal instability pathological metric is computed from DNA from the biological sample of the training histological image. In some embodiments, the chromosomal pathological metric and the biological sample of the training histological image come from the same individual (e.g., a patient). In some embodiments, the DNA from which the corresponding chromosomal pathological metric is computed is from the same patient as the biological sample of the training histological image. In some embodiments, the corresponding chromosomal instability pathological metric is matched with a training histological image. The image is segmented into individual tiles of the training histological image, 202. These training histological image tiles and matched chromosomal instability pathological metrics corresponding to the training histological images can be used to train the machine-learning models in a system described herein, which are overlaid 203.

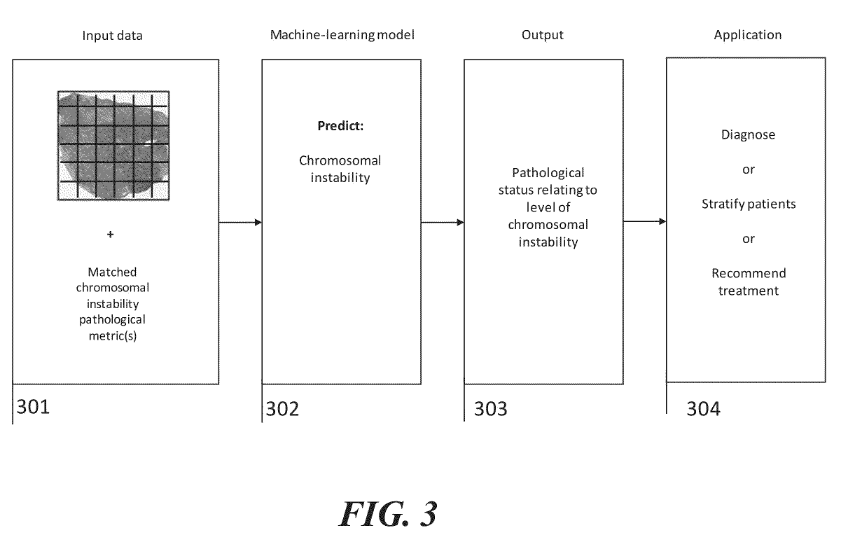

One aspect of the present application provides systems for using the methods described herein to characterize a disease in a patient using input histological images and a trained machine-learning model, wherein the machine-learning model is trained according to the machine-learning model training methods described herein. FIG. 3 illustrates a system for characterizing a disease in a patient with the methods described herein. A histological image (e.g., an input histological image) or tiles of a histological image (e.g., input histological image segments) are obtained from a patient tissue biopsy biological sample and are inputted into the trained machine-learning model, 301. In some embodiments, an H&E stained histological image (e.g., an input histological image) or tiles of an H&E stained histological image (e.g., input histological image segments) are obtained from a patient tissue biopsy biological sample and are inputted into the trained machine-learning model, 301. The machine-learning model predicts the chromosomal instability (e.g., a predicted pathological status metric, such as the predicted pathological status metric is a probability of high chromosomal instability of the biological sample in the input histological image or input histological image tiles, a continuous chromosomal instability score of the biological sample in the input histological image or input histological image tiles, a binary classification of high or low chromosomal instability of the biological sample in the input histological image or input histological image tiles, or any combination thereof) of the input histological image or input histological image tiles, 302. The machine-learning model also assigns a probability of chromosomal instability for use in the model output, 302.

The machine-learning model is trained to output a predicted pathological status metric, such as the types of pathological status metrics in the input histological image or input histological image tiles, correlated to the chromosomal instability in the input histological image or input histological image segments, 303. The output is displayed to a user who can use the pathological status metrics related to chromosomal instability for a variety of tasks such as, to diagnose a patient, stratify multiple patients based on the level of chromosomal instability identified in their biological samples, characterize disease status, and/or to recommend specific treatments, diagnose, or understand prognosis, as described herein, 304.

One aspect of the present application provides systems for implementing the methods described herein. In some embodiments, the disclosed systems may comprise one or more processors or computer system, one or more memory devices, and one or many programs, where the one or more programs are stored in the memory devises and contain instructions which, when executes by one or more processors, cause the system to perform the method for training a machine-learning model to detect chromosomal instability from input histological images, or image tiles thereof, or the method for characterizing a disease using a machine-learning model able to detect chromosomal instability from input histological images, or image tiles thereof, as described herein. In some aspects, the disclosed system may further comprise one or more display devices (e.g., monitors), one or more imaging units (e.g., bright-field microscope, florescent microscope), one or more output devices (e.g., printers), one or more computer network interface devices, or any combination thereof.

In some embodiments, the performance of the disclosed methods and systems may be assessed. In some embodiments, assessing the methods and systems may include determining the area under a receiver operating characteristic curve (AUROC) on either a per histological image basis or after the aggregation of results from multiple histological images or histological image segments. A receiver operating characteristic (ROC) curve is a graphical plot of the classification model’s performance as its discrimination threshold is varied. In some instances, the performance of the disclosed methods and systems may be characterized by an AUROC value of at least 0.50, at least 0.55, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some instances, the performance of the disclosed methods and systems may be characterized by an AUROC of any value within the range of values described in this paragraph, e.g., an AUROC value of 0.876. In some instances, the performance of the disclosed methods and systems may vary depending on the specific training histological images for which the classification models are trained.

In some embodiments, the performance of the disclosed methods and systems may be assessed by evaluating the clinical sensitivity and specificity for correctly identifying a pathological status related to a level of chromosomal instability in an input histological images, or image tiles thereof. In some instances, the clinical sensitivity (e.g., how often the method correctly identifies the pathological status related to the level of chromosomal instability, as calculated from the number of true positive results divided by the sum of true positive and false negative results), may be at least 50%, at least 60%, at least 70%, at least 75%, at least 80%, at least 85%, at least 90%, at least 95%, at least 98%, at least 99%, or at least 99.9%. In some instances, the clinical specificity (e.g., how often the method correctly identifies the pathological status related to the level of chromosomal instability, as calculated from the number of true negatives divided by the sum of false positives and true negatives) may be at least 50%, at least 60%, at least 70%, at least 75%, at least 80%, at least 85%, at least 90%, at least 95%, at least 98%, at least 99%, or at least 99.9%. In some instances, adjustment of a threshold used to distinguish between positive and negative results may result in tradeoffs between clinical sensitivity and clinical specificity. For example, the threshold may be adjusted to increase clinical sensitivity with a concomitant decrease in clinical specificity, or vice versa.

In some embodiments, the performance of the disclosed methods and systems may be assessed by the positive predictive value (PPV) of the disclosed methods and systems (i.e., the percentage of positive results that are true positives as indicated by a pathologist annotation or other reference method) is calculated as the number of true positives divided by the sum of true positives and false positives. The PPV may be at least 70%, at least 75%, at least 80%, at least 85%, at least 90%, at least 95%, at least 98%, at least 99%, or at least 99.9%. In some embodiments, the negative predictive value (NPV) of the disclosed methods and systems (e.g., the percentage of negative results that are true negatives as indicated by a pathologist annotation or other reference method) is calculated from the number of true negatives divided by the sum of false negatives and true negatives can be used to assess the model performance. The NPV may be at least 70%, at least 75%, at least 80%, at least 85%, at least 90%, at least 95%, at least 98%, at least 99%, or at least 99.9%.

### V. Applications of the Methods

In some aspects, the provided embodiments can be applied in a method for characterizing a disease in an individual, such as characterizing a cancer, for example from intact tissues or samples (e.g., a tumor resection) in which a chromosomal instability metric has been determined using the provided machine-learning models. In some aspects, the embodiments can be applied to a diagnostic or prognostic method. In some aspects, the provided embodiments can be used to inform treatment selection (e.g., selection of a pharmaceutical drug) and/or evaluate the efficacy of a treatment, to further characterize a disease (e.g., cancer) in the individual.

In some embodiments, the individual is a mammal (e.g., human, non-human primate, rat, mouse, cow, horse, pig, sheep, goat, dog, cat, etc.). In some embodiments, the individual is a human. The individual may be a clinical patient, a clinical trial volunteer, or an experimental animal. In some embodiments, the individual is younger than about 60 years old (including, for example, younger than about any of 50, 40, 30, 25, 20, 15, 10 years old, or younger). In some embodiments, the individual is older than about 60 years old (including, for example, older than about any of 70, 80, 90, 100 years old, or older). In some embodiments, the individual is diagnosed with, or genetically prone to, one or more of the diseases or disorders described herein (such as a cancer). In some embodiments, the individual has one or more risk factors associated with one or more diseases or disorders described herein. For example, the individual may be a patient suspected of having a disease, such as cancer. In some embodiments, the patient has been previously diagnosed with a disease prior to the application of a provided machine-learning model. In some embodiments, the patient has been previously diagnosed with cancer prior to the application of a provided machine-learning model.

A disease is characterized based on the determined chromosomal instability pathological metric obtained using a machine-learning model described herein. In some embodiments, input histological images or input histological image segments of a biological sample (e.g., a tumor sample from a patient) are inputted into a trained machine-learning model trained using a plurality of training histological images and one or more matched chromosomal instability pathological metrics corresponding to the plurality of training histological images. The pathological status of the one or more input histological images may be classified using the trained machine-learning model described herein. In some embodiments, the computed pathological status is described as a pathological status metric, and is related to the chromosomal instability for the biological sample of the input histological images. The pathological status metric related to chromosomal instability is then used to characterize a disease in the patient.

The disease characterized using information (e.g., a chromosomal instability metric) obtained from a provided machine-learning model may include, but is not limited to, cancer, autoimmune disease, central nervous system (CNS) disease, Fanconi anaemia, Nijmegen breakage syndrome, Bloom syndrome, ataxia telangiectasia, ataxia telangiectasia-like disorder, immunodeficiency/centromeric instability/facial anomalies syndrome, Cockayne syndrome, trichothiodystrophy, xeroderma pigmentosum, DNA ligase I deficiency, PMS2 deficiency, and DNA recombinase repair defects (e.g., DNA-PKcs, Artemis, DNA ligase 4, Cernunnos/XLF). In some embodiments, the cancer includes, but is not limited to, acute lymphoblastic leukemia (ALL), acute myeloid leukemia (AML), bladder cancer, breast cancer, brain cancer, cervical cancer, colon cancer, colorectal cancer, endometrial cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, prostate cancer, squamous cell carcinoma, testicular cancer, stomach cancer, or thyroid cancer. In some instances, a cancer includes a lymphoid neoplasia, head and neck cancer, pancreatic cancer, endometrial cancer, colon or colorectal cancer, prostate cancer, glioma or other brain/spinal cancers, ovarian cancer, lung cancer, bladder cancer, melanoma, breast cancer, a myeloid neoplasia, testicular cancer, stomach cancer, cervical, kidney, liver, or thyroid cancer.

In some embodiments, the provided methods are applicable to a diagnostic method for diagnosis of a disease in a patient. For example, a provided machine-learning model may be used to diagnose cancer in the patient, wherein the chromosomal instability metric may be used to diagnose and/or predict the occurrence of a disease in the patient. In some embodiments, the pathological status metric of the input histological image or input histological image segments are indicative of a disease profile in the patient when the chromosomal instability of the input histological image is above a specified threshold. For example, a cancer disease profile may comprise an increased bulk genomic aneuploidy burden, increased fraction of the genome altered (FGA), increased activity of cyclic GMP-AMP synthase (cGAS)-stimulator of interferon genes (STING), higher levels of CIN23 gene signature, a gain/loss of chromosomes, increased copy number heterogeneity, increased type I interferon expression, increased presence of micronuclei, etc. in an input histological image or input histological image segments. In some embodiments, the patient is diagnosed with a cancer based on the pathological status metric related to chromosomal instability of the input histological images determined using a provided machine-learning model.

In some embodiments, the chromosomal instability metric, determined using a provided machine-learning model, is used for predicting disease prognosis in a patient. Disease prognosis is determined in patients that have already developed the disease of interest, such as cancer. The term “disease prognosis” as used herein refers to a prediction of the possible outcomes of a disease (e.g., death, chance of recovery, and/or recurrence) and the frequency with which these outcomes can be expected to occur. The prognosis of a disease may be predicted based on the degree of disease progression in the patient. In some embodiments, the disease is cancer, and a cancer prognosis is predicted in a patient using a chromosomal instability metric determined using a machine-learning model described herein. Additional factors contributing to disease prognosis, such as demographics (e.g., age), behavior (e.g., alcohol consumption, smoking), and co-morbidity (e.g., other conditions accompanying the disease in question), may be evaluated in combination with the chromosomal instability metric. Cancer-specific factors contributing to disease prognosis in combination with the chromosomal instability metric may include, but are not limited to, tumor size, high white cell count, cancer type, and the location of the cancer within the patient’s body.

In some cases, the disease (e.g., cancer) is further classified by a grade for prognostic purposes. For example, the grades of cancer include Grade X, Grade 1, Grade 2, Grade 3 or Grade 4. In some embodiments, the cancer grade is determined based on the chromosomal instability metric of the input histological images. In some embodiments, the cancer grade is further indicated by a category of tubule formation, nuclear grade and/or the mitotic rate. In some cases, a cancer stage is assigned based on the tumor, the regional lymph nodes and/or distant metastasis. For example, the stages assigned to the tumor include TX, T0, Tis, T1, T2, T3, or T4. For example, the stages assigned to a regional lymph node include NX, N0, N1, N2, or N3. For example, the stages assigned to a distant metastasis include MX, M0, or M1. In some embodiments, the stages include stage 0, stage I, stage II, stage III or stage IV. Sometimes, the cancer is classified as more than one grade or stage of cancer. In some embodiments, a chromosomal instability metric can be correlated to different stages of tumor development, such as initiation, promotion, and progression. In some embodiments, the grade and stage of the cancer is used to predict disease prognosis in combination with the chromosomal instability metric determined using a provided machine-learning model.

In some embodiments, the method further comprises implementing a treatment regimen based on the disease diagnosis and/or prognosis of the disease. In some embodiments, the treatment regimen comprises treatment with a drug that specifically targets chromosomal instability in a patient. In some embodiments, the drug is a pharmaceutical composition. For example, the drug may target abnormal chromosomal structures. In some embodiments, drugs with various mechanisms of action, such as anti-microtubule activity, histone deacetylase inhibition, mitotic checkpoint inhibition, and targeting of DNA replication and damage responses, may serve as effective treatment for chromosomal instability. Drugs effective for the treatment of a cancer described herein include proteasome inhibitors, immunomodulatory drugs, glucocorticoids, and conventional chemotherapeutics. In some embodiments, drug treatment is combined with autologous stem cell transplantation if the patient is eligible.

The amount of the drug (e.g., pharmaceutical composition) administered to a patient may vary with the particular composition, the method of administration, and the particular type of disease being treated. The amount should be sufficient to produce a desirable beneficial effect. For example, in some embodiments, the amount of the drug is effective to result in an objective response (such as a partial response or a complete response). In some embodiments, the amount of drug is sufficient to result in a complete response in the individual. In some embodiments, the amount of the drug is sufficient to result in a partial response in the individual. In some embodiments, the amount of the drug administered alone is sufficient to produce an overall response rate of more than about any one of 40%, 50%, 60%, or 64% among a population of patients treated with the composition. Responses of the patients to the treatment can be determined, for example, based on the chromosomal instability metric of the methods described herein.

In another embodiment, described herein are methods for determining the therapeutic efficacy of a pharmaceutical drug. These methods are useful in informing choice of the drug for a patient, as well as monitoring the progress of a patient on the drug.

Treatment of a patient involves administering the drug, or a combination of drugs, in a particular regimen. In some instances, the regimen involves a single dose of the drug or multiple doses of the drug, or a combination of drugs, over time. The doctor or clinical researcher monitors the effect of the drug on the patient or subject over the course of administration. If the drug has a pharmacological impact on the condition, the profile of a chromosomal instability metric of the biological sample of the input histological images of the present invention are changed toward a non-cancer profile (e.g., decreased levels of pathological status metric related to chromosomal instability). In some embodiments, the non-cancer profile comprises a decreased frequency of chromosomal instability in an input histological image or input histological image segments.

In some embodiments, the drug is administered in an amount sufficient to decrease the size of a tumor, decrease the number of cancer cells, or decrease the growth rate of a tumor by at least about any one of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing. In some embodiments, the drug is administered in an amount sufficient to decrease a pathological status metric related to chromosomal instability in a histological image by at least about any one of 10%, 20%, 30%, 40%, 50%, 60%, 70%, 80%, 90%, 95% or 100% compared to the corresponding level of the pathological status metric related to chromosomal instability in the same subject prior to treatment or compared to the corresponding activity in other subjects not receiving the treatment.

In some instances, the chromosomal instability metric in the patient is followed during the course of treatment. In some embodiments, the patient is re-biopsied during the course of treatment (e.g., during the course of a clinical trial) and the new biopsy sample is used in a method of characterizing a disease described herein. Accordingly, this method involves determining the chromosomal instability metric in histological images from a patient receiving drug therapy, and correlating the levels with the cancer status of the patient (e.g., by comparison to predefined chromosomal instability metrics that correspond to different cancer statuses). The effect of therapy is determined based on these comparisons. If a treatment is effective, then the chromosomal instability metric of the histological images trend toward normal, while if treatment is ineffective, the chromosomal instability metric of the histological images trend toward cancer indications.

In some embodiments, the methods described herein are used to stratify patients with a disease (e.g., cancer) based on chromosomal instability for the clinical applications described herein. For example, the methods could be performed on a plurality of histological images from multiple patients and the resulting chromosomal instability metrics can be used to separate patients into different diagnostic or treatment groups.

### EXEMPLARY EMBODIMENTS

The following embodiments are exemplary and are not intended to limit the scope of the invention described herein.

Embodiment 1. A method for characterizing a disease in a patient, comprising: inputting one or more input histological images of a biological sample into a machine-learning model, wherein the machine learning model is trained using a plurality of training histological images and one or more matched chromosomal instability pathological metrics corresponding to the plurality of training histological images; and, classifying a pathological status of the biological sample in the one or more input histological images using the trained machine-learning model.

Embodiment 2. The method of embodiment 1, wherein the biological sample comprises at least a portion of a solid tumor.

Embodiment 3. The method of embodiment 2, wherein the at least a portion of the solid tumor is a biopsy slice of a solid tumor.

Embodiment 4. The method of any one of embodiments 1-3, wherein the biological sample relates to a plurality of training or input histological images from the same patient.

Embodiment 5. The method of any one of embodiments 1-4, wherein the matched chromosomal pathological metric is obtained from DNA from the biological sample of the training histological image.

Embodiment 6. The method of any one of embodiments 1-5, wherein the matched chromosomal pathological metric is computed from DNA from the same patient as the biological sample of the training histological image.

Embodiment 7. The method of any one of embodiments 1-6, wherein the matched chromosomal pathological metric and the biological sample of the training histological image come from the same patient.

Embodiment 8. The method of any one of embodiments 1-7, wherein the one or more input histological images and/or the plurality of training histological images is an image captured between 2.5 x and 20 x magnification.

Embodiment 9. The method of any one of embodiments 1-8, wherein the one or more input histological images and/or the plurality of training histological images is captured at a resolution between 256 pixel x 256 pixel and 10,000 pixel x 10,000 pixel.

Embodiment 10. The method of any one of embodiments 1-9, wherein the one or more input histological images and/or the plurality of training histological images are hematoxylin and eosin (H&E) and/or 4′,6-diamidino-2-phenylindole (DAPI) stained images.

Embodiment 11. The method of any one of embodiments 1-10, further comprising segmenting one or more whole images into a plurality of image tiles, wherein the image tiles are inputted into the machine-learning model as the input histological images and/or the training histological images.

Embodiment 12. The method of any one of embodiments 1-10, wherein the machine-learning model segments the input histological images and/or the training histological images into tiles.

Embodiment 13. The method of any one of embodiments 1-12, wherein the one or more of the input histological images and/or the plurality of training images are deposited into computer cloud storage.

Embodiment 14. The method of any one of embodiments 1-13, wherein the machine-learning model is an unsupervised model.

Embodiment 15. The method of any one of embodiments 1-13, wherein the machine-learning model is a weakly-supervised model.

Embodiment 16. The method of any one of embodiments 1-13, wherein the machine-learning model is a human-in-the-loop model.

Embodiment 17. The method of any one of embodiments 1-16, wherein the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

Embodiment 18. The method of any one of embodiments 1-17, wherein the matched chromosomal instability pathological metrics and the training histological images are used to predict the pathological status of the input histological images.

Embodiment 19. The method of embodiment 18, wherein the pathological status is described as a metric, wherein the pathological status metric is selected from the group consisting of a probability of high chromosomal instability in the image, a continuous chromosomal instability score, and a binary classification of high or low chromosomal instability.

Embodiment 20. The method of embodiment 19, wherein the pathological status metric is displayed to a user.

Embodiment 21. The method of any one of embodiments 1-20, wherein the matched chromosomal instability pathological metrics are displayed to a user.

Embodiment 22. The method of any one of embodiments 1-21, wherein characterizing a disease comprises diagnosing the disease.

Embodiment 23. The method of any one of embodiments 1-21, wherein characterizing a disease comprises informing a treatment strategy.

Embodiment 24. The method of any one of embodiments 1-21, wherein characterizing a disease comprises evaluating the disease progression.

Embodiment 25. The method of any one of embodiments 1-21, wherein characterizing a disease comprises predicting the disease prognosis.

Embodiment 26. The method of any one of embodiments 1-21, wherein characterizing a disease comprises evaluating effect of a treatment.

Embodiment 27. The method of any one of embodiments 1-21, wherein characterizing a disease comprises identifying a patient population for treatment.

Embodiment 28. The method of any one of embodiments 1-27, wherein the disease is a cancer.

Embodiment 29. The method of any one of embodiments 1-28, wherein the method is implemented on a cloud-based computing platform.

Embodiment 30. A system for characterizing a disease in a patient with machine-learning, comprising: one or more processors; a memory; and one or more programs with instructions for: receiving data representing one or more input histological images of a biological sample; and, classifying a pathological status of the biological sample in the one or more input histological images using a trained machine-learning model trained using a plurality of training histological images and one or more matched chromosomal instability pathological metrics corresponding to the plurality of training histological images.

Embodiment 31. The system of embodiment 30, wherein the biological sample comprises at least a portion of a solid tumor.

Embodiment 32. The system of embodiment 31, wherein the at least a portion of the solid tumor is a biopsy slice of a solid tumor.

Embodiment 33. The system of any one of embodiments 30-32, wherein the biological sample relates to a plurality of training or input histological images from the same patient.

Embodiment 34. The system of any one of embodiments 30-33, wherein the matched chromosomal pathological metric is obtained from DNA from the biological sample of the training histological image.

Embodiment 35. The system of any one of embodiments 30-34, wherein the matched chromosomal pathological metric is computed from DNA from the same patient as the biological sample of the training histological image.

Embodiment 36. The system of any one of embodiments 30-36, wherein the matched chromosomal pathological metric and the biological sample of the training histological image come from the same patient.

Embodiment 37. The system of any one of embodiments 30-36, wherein the one or more input histological images and/or the plurality of training histological images is an image captured between 2.5 x and 20 x magnification.

Embodiment 38. The system of any one of embodiments 30-37, wherein the one or more input histological images and/or the plurality of training histological images is captured at a resolution between 256 pixel x 256 pixel and 10,000 pixel x 10,000 pixel.

Embodiment 39. The system of any one of embodiments 30-38, wherein the one or more input histological images and/or the plurality of training histological images are hematoxylin and eosin (H&E) and/or 4′,6-diamidino-2-phenylindole (DAPI) stained images.

Embodiment 40. The system of any one of embodiments 30-38, wherein the instructions further comprise instructions for segmenting one or more whole images into a plurality of image tiles, wherein the image tiles are inputted into the machine-learning model as the input histological images and/or the training histological images.

Embodiment 41. The system of any one of embodiments 30-38, wherein the machine-learning model segments the input histological images and/or the training histological images into tiles.

Embodiment 42. The system of any one of embodiments 30-41, wherein the one or more input histological images and/or the plurality of training histological images are deposited into a computer cloud.

Embodiment 43. The system of any one of embodiments 30-42, wherein the machine-learning model is an unsupervised model.

Embodiment 44. The system of any one of embodiments 30-42, wherein the machine-learning model is a weakly-supervised model.

Embodiment 45. The system of any one of embodiments 30-42, wherein the machine-learning model is a human-in-the-loop model.

Embodiment 46. The system of any one of embodiments 30-45, wherein the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

Embodiment 47. The system of any one of embodiments 30-46, wherein the matched chromosomal instability pathological metric and training histological images are used to predict the pathological status of the input histological images.

Embodiment 48. The system of embodiment 47, wherein the pathological status is described as a metric, wherein the pathological status metric is selected from the group consisting of a probability of high chromosomal instability in the image, a continuous chromosomal instability score, and a binary classification of high or low chromosomal instability.

Embodiment 49. The system of any one of embodiments 48, wherein the pathological status metric is displayed to a user.

Embodiment 50. The system of any one of embodiments 30-49, wherein the matched chromosomal instability pathological metrics are displayed to a user.

Embodiment 51. The system of any one of embodiments 30-50, wherein characterizing a disease comprises diagnosing the disease.

Embodiment 52. The system of any one of embodiments 30-50, wherein characterizing a disease comprises informing a treatment strategy.

Embodiment 53. The system of any one of embodiments 30-50, wherein characterizing a disease comprises evaluating the disease progression.

Embodiment 54. The system of any one of embodiments 30-50, wherein characterizing a disease comprises predicting the disease prognosis.

Embodiment 55. The system of any one of embodiments 30-50, wherein characterizing a disease comprises evaluating effect of a treatment.

Embodiment 56. The system of any one of embodiments 30-50, wherein characterizing a disease comprises identifying a patient population for treatment.

Embodiment 57. The system of any one of embodiments 30-56, wherein the disease is a cancer.

Embodiment 58. The system of any one of embodiments 30-57, wherein the instructions are implemented on a cloud-based computing platform.

Embodiment 59. The system of any one of embodiments 30-58, wherein the instructions for implementing the instructions reside in cloud storage.

Embodiment 60. A method for training a machine-learning model to analyze histological images of biological samples, comprising: obtaining a chromosomal instability pathological metric for each training histological image of a plurality of training histological images; and training the machine-learning model based on the plurality of training histological images and the matched calculated chromosomal instability pathological metrics, wherein the machine-learning model is trained to receive one or more input histological images and output a pathological status of the one or more input histological image.

Embodiment 61. The method of embodiment 60, wherein the biological samples comprises at least a portion of a solid tumor.

Embodiment 62. The method of embodiment 61, wherein the at least a portion of the solid tumor is a biopsy slice of a solid tumor.

Embodiment 63. The method of any one of embodiments 60-62, wherein the biological sample relates to a plurality of training or input histological images from the same patient.

Embodiment 64. The method of any one of embodiments 60-63, wherein the matched chromosomal pathological metric is obtained from DNA from the biological sample of the training histological image.

Embodiment 65. The method of any one of embodiments 60-64, wherein the matched chromosomal pathological metric is computed from DNA from the same patient as the biological sample of the training histological image.

Embodiment 66. The method of any one of embodiments 60-65, wherein the matched chromosomal pathological metric and the biological sample of the training histological image come from the same patient.

Embodiment 67. The method of any one of embodiments 60-66, wherein the one or more input histological images and/or the plurality of training histological images is an image captured between 2.5 x and 20 x magnification.

Embodiment 68. The method of any one of embodiments 60-67, wherein the one or more input histological images and/or the plurality of training histological images is captured at a resolution between 256 pixel x 256 pixel. and 10,000 pixel x 10,000 pixel.

Embodiment 69. The method of any one of embodiments 60-68, wherein the one or more input histological images and/or the plurality of training histological images are hematoxylin and eosin (H&E) and/or 4′,6-diamidino-2-phenylindole (DAPI) stained images.

Embodiment 70. The method of any one of embodiments 60-69, wherein the matched chromosomal instability pathological metric describes the chromosomal instability of the biological sample.

Embodiment 71. The method of embodiment 70, wherein the matched chromosomal instability pathological metric is a fraction of genome altered.

Embodiment 72. The method of embodiment 71, wherein the fraction of genome altered is calculated using DNA sequencing data.

Embodiment 73. The method of any one of embodiments 60-72, further comprising segmenting one or more whole images into a plurality of image tiles, wherein the image tiles are inputted into the machine-learning model as the input histological images and/or the training histological images.

Embodiment 74. The method of any one of embodiments 60-72, wherein the machine-learning model segments the input histological images and/or the training histological images into tiles.

Embodiment 75. The method of any one of embodiments 60-74 wherein the one or more of the input histological images and/or the plurality of training histological images are deposited into a computer cloud.

Embodiment 76. The method of any one of embodiments 60-75, wherein the machine-learning model is an unsupervised model.

Embodiment 77. The method of any one of embodiments 60-75, wherein the machine-learning model is a weakly- supervised model.

Embodiment 78. The method of any one of embodiments 60-75, wherein the machine-learning model is a human-in-the-loop model.

Embodiment 79. The method of any one of embodiments 60-78, wherein the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

Embodiment 80. The method of any one of embodiments 60-79, wherein the matched chromosomal instability pathological metric and training histological images are used to predict the pathological status of the input histological images.

Embodiment 81. The method of embodiment 80, wherein the pathological status is described as a metric, wherein the pathological status metric is selected from the group consisting of a probability of high chromosomal instability in the image, a continuous chromosomal instability score, and a binary classification of high or low chromosomal instability.

Embodiment 82. The method of embodiment 81, wherein the pathological status metric is displayed to a user.

Embodiment 83. The method of any one of embodiments 60-82, wherein the matched chromosomal instability pathological metrics are displayed to a user.

Embodiment 84. The method of any one of embodiments 60-83, wherein the method is implemented on a cloud-based computing platform.

Embodiment 85. A system for training a machine-learning model to predict a pathological status, comprising one or more processors; a memory; and one or more programs with instructions for: receiving a plurality of chromosomal instability pathological metrics for a plurality of training histological images by calculating the chromosomal instability pathological metrics in the plurality of training histological images; training the machine-learning model based on the plurality of training histological images and the matched calculated chromosomal instability pathological metrics, wherein the machine-learning model is trained to receive one or more input histological images and output a pathological status of the one or more input histological images.

Embodiment 86. The system of embodiment 85, wherein biological sample comprises at least a portion of a solid tumor.

Embodiment 87. The system of embodiment 86, wherein the at least a portion of the solid tumor is a biopsy slice of a solid tumor.

Embodiment 88. The system of any one of embodiments 85-87, wherein the biological sample relates to a plurality of training or input histological images from the same patient.

Embodiment 89. The system of any one of embodiments 85-88, wherein the matched chromosomal pathological metric is obtained from DNA from the biological sample of the training histological image.

Embodiment 90. The system of any one of embodiments 85-89, wherein the matched chromosomal pathological metric is computed from DNA from the same patient as the biological sample of the training histological image.

Embodiment 91. The method of any one of embodiments 85-90, wherein the matched chromosomal pathological metric and the biological sample of the training histological image come from the same patient.

Embodiment 92. The system of any one of embodiments 85-91, wherein the one or more input histological images and/or the plurality of training histological images is an image captured between 2.5 x and 20 x magnification.

Embodiment 93. The system of any one of embodiments 85-92, wherein the one or more input histological images and/or the plurality of training histological images is captured at a resolution between 256 pixel x 256 pixel and 10,000 pixel x 10,000 pixel.

Embodiment 94. The system of any one of embodiments 85-93, wherein the one or more input histological images and/or the plurality of training histological images are hematoxylin and eosin (H&E) and/or 4′,6-diamidino-2-phenylindole (DAPI) stained images.

Embodiment 95. The system of any one of embodiments 85-94, wherein the chromosomal instability pathological metric describes the chromosomal instability of the biological sample.

Embodiment 96. The system of embodiment 95, wherein the chromosomal instability pathological metric is a fraction of genome altered.

Embodiment 97. The system of embodiment 96, wherein the fraction of genome altered is calculated using DNA sequencing data.

Embodiment 98. The system of any one of embodiments 85-98, wherein the instructions further comprise instructions for segmenting one or more whole images into a plurality of image tiles, wherein the image tiles are inputted into the machine-learning model as the input histological images and/or the training histological images.

Embodiment 99. The system of any one of embodiments 85-97, wherein the machine-learning model segments the input histological images and/or the training histological images into tiles.

Embodiment 100. The system of any one of embodiments 85-99, wherein the one or more input histological images and/or the plurality of training histological images are deposited into a computer cloud.

Embodiment 101. The system of any one of embodiments 87-100, wherein the machine-learning model is an unsupervised model.

Embodiment 102. The system of any one of embodiments 87-100, wherein the machine-learning model is a weakly-supervised model.

Embodiment 103. The system of any one of embodiments 85-100, wherein the machine-learning model is a human-in-the-loop model.

Embodiment 104. The system of any one of embodiments 85-103, wherein the machine-learning model applies a model selected from Support Vector Machines (SVM), Random Forests(RF), Artificial Neural Network (ANN), Convolutional Neural Network (CNN), K-means, ResNet, DenseNet, and eXtreme Gradient Boosting (XGBoost).

Embodiment 105. The system of any one of embodiments 85-104, wherein the matched chromosomal instability pathological metrics and training histological images are used to predict the pathological status of the input histological image.

Embodiment 106. The system of embodiment 105, wherein the pathological status is described as a metric, wherein the pathological status metric is selected from the group consisting of a probability of high chromosomal instability in the image, a continuous chromosomal instability score, and a binary classification of high or low chromosomal instability.

Embodiment 107. The system of embodiment 106, wherein the pathological status metric is displayed to a user.

Embodiment 108. The system of any one of embodiments 85-107, wherein the matched chromosomal instability pathological metrics are displayed to a user.

Embodiment 109. The system of any one of embodiments 85-108, wherein the instructions are implemented on a cloud-based computing platform.

Embodiment 110. A non-transitory computer-readable storage medium storing one or more programs, the one or more programs comprising instructions, which when executed by one or more processors of an electronic device having a display, cause the electronic device to: receive one or more input histological images of a biological sample; input one or more input histological images of a biological sample into a machine-learning model trained using a plurality of training histological images and one or more matched chromosomal instability pathological metrics corresponding to the plurality of training histological images; and, classify a pathological status of the biological sample in the one or more input histological images using the training machine-learning model.

Embodiment 111. The computer-readable storage medium of embodiment 110, wherein the biological sample comprises at least a portion of a solid tumor.

Embodiment 112. The computer-readable storage medium of embodiment 111, wherein the at least a portion of the solid tumor is a biopsy slice of a solid tumor.

Embodiment 113. The computer-readable storage medium of any one of embodiments 110-112, wherein the biological sample relates to a plurality of training or input histological images from the same patient.

Embodiment 114. The computer-readable storage medium of any one of embodiments 1140113, wherein the matched chromosomal pathological metric is obtained from DNA from the biological sample of the training histological image.

Embodiment 115. The computer-readable storage medium of any one of embodiments 110-114, wherein the matched chromosomal pathological metric is computed from DNA from the same patient as the biological sample of the training histological image.

Embodiment 116. The computer-readable storage medium of any one of embodiments 110-115, wherein the matched chromosomal pathological metric and the biological sample of the training histological image come from the same patient.

Embodiment 117. The computer-readable storage medium of any one of embodiments 110-116, wherein the one or more input histological images and/or the plurality of training histological images is an image captured between 2.5 x and 20 x magnification.

Embodiment 118. The computer-readable storage medium of any one of embodiments 110-117, wherein the one or more input histological images and/or the plurality of training histological images is captured at a resolution between 256 pixel x 256 pixel and 10,000 pixel x 10,000 pixel.

Embodiment 119. The computer-readable storage medium of any one of embodiments 110-118, wherein the one or more input histological images and/or the plurality of training histological images are hematoxylin and eosin (H&E) and/or 4′,6-diamidino-2-phenylindole (DAPI) stained images.

Embodiment 120. The computer-readable storage medium of any one of embodiments 110-119, wherein the matched chromosomal instability pathological metric describes the chromosomal instability of the biological sample.

Embodiment 121. The computer-readable storage medium of embodiment 120, wherein the matched chromosomal instability pathological metric is a fraction of genome altered.

Embodiment 122. The computer-readable storage medium of embodiment 121, wherein the fraction of genome altered is calculated using DNA sequencing data.

Embodiment 123. The computer-readable storage medium of any one of embodiments 110-122, wherein the instructions further comprise instructions for segmenting one or more whole images into a plurality of image tiles, wherein the image tiles are inputted into the machine-learning model as the input histological images and/or the training histological images.

Embodiment 124. The computer-readable storage medium of any one of embodiments 110-122, wherein the machine-learning model segments the input histological images and/or the training histological images into tiles.

Embodiment 125. The computer-readable storage medium of any one of embodiments 110-124, wherein the machine-learning model is an unsupervised model.

Embodiment 126. The computer-readable storage medium of any one of embodiments 110-124, wherein the machine-learning model is a weakly-supervised model.

Embodiment 127. The computer-readable storage medium of any one of embodiments 110-124, wherein the machine-learning model is a human-in-the-loop model.

Embodiment 128. The computer-readable storage medium of any one of embodiments 110-127, wherein the machine-learning model applies a model selected from Support Vector Machines (SVM), Random Forests (RF), Artificial Neural Network (ANN), Convolutional Neural Network (CNN), K-means, ResNet, DenseNet, and eXtreme Gradient Boosting (XGBoost).

Embodiment 129. The computer-readable storage medium of any one of embodiments 110-128, wherein the matched chromosomal instability pathological metrics and training histological images are used to predict the pathological status of the one or more input histological images.

Embodiment 130. The computer-readable storage medium of embodiment 129, wherein the pathological status is described as a metric, wherein the pathological status metric is selected from the group consisting of a probability of high chromosomal instability in the image, a continuous chromosomal instability score, and a binary classification of high or low chromosomal instability.

Embodiment 131. The computer-readable storage medium of embodiment 130, wherein the pathological status metric is displayed to a user.

Embodiment 132. The computer-readable storage medium of any one of embodiments 110-131, wherein the matched chromosomal instability pathological metrics are displayed to a user.

Embodiment 133. The computer-readable storage medium of any one of embodiments 110-132, wherein the one or more computer programs are implemented on a cloud-based computing platform.

Embodiment 134. The computer-readable storage medium of any one of embodiments 110-133, wherein the instructions for implementing the one or more computer programs reside in cloud storage.

### EXAMPLES

The examples below are intended to be purely exemplary of the invention and should therefore not be considered to limit the invention in any way. The following examples and detailed description are offered by way of illustration and not by way of limitation.

### Example 1: Training a Machine-Learning Model Using Biological Images From the Cancer Genome Atlas (Tcga) and Matched Fraction of Genome Altered Statistics

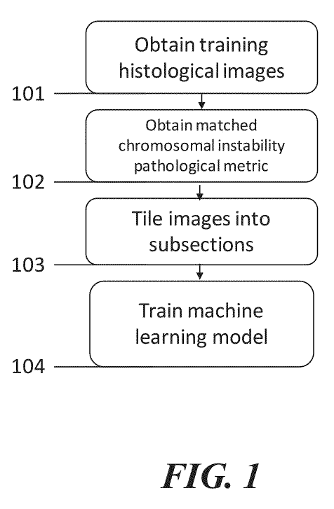

This example demonstrates a method for training a machine-learning model that can predict a pathological status of from histological images (e.g., stained input histological images, such as hematoxylin and eosin (H&E) stained or DAPI stained input histological images) of tumor samples (FIG. 1). In particular, this example includes training a convolutional neural network (CNN) model with data downloaded from The Cancer Genome Atlas (TCGA). Specifically, the model is trained with H&E stained training histological images downloaded from the TCGA database (101) and matched chromosomal instability pathological metrics (such as fraction of genome altered (FGA) metrics) that have been calculated based on the DNA sequences that accompany (e.g., are matched with and correspond to) each sample (103), such as each from H&E stained training histological image, downloaded from TCGA. FGA has been previously shown to be correlated with chromosomal instability of a biological sample.

The H&E stained training histological images are images of primary tumors from a biological sample obtained from a patient having cancer, and are downloaded from the TCGA database (101). The tumor type that may be used for the training methods described herein are not limited, such that the method is application to a wide variety of cancer types and cancer stages. The training histological images have been captured at between 2.5 x and 10 x magnification.

Each training histological image is subsectioned into segments before machine-learning model training (102). In some embodiments, the machine-learning model training further comprises subsectioning training histological images into segments. A sliding window approach may be used to identify 256 pixel x 256 pixel segments of each whole slide histological image with greyscale values greater than a threshold value. The threshold is chosen based on the greyscale value that differentiates the background image from the biological sample. The matched chromosomal instability pathological metric (e.g., FGA metric) is computed as the sum of altered genome size/total genome size analyzed, using whole exome sequencing and copy number variation (CNV) data (103).

The training histological image segments and matched chromosomal instability pathological metrics (e.g., FGA metrics) are used to train a CNN machine-learning model, wherein the machine-learning model is trained to receive a histological image (e.g., an input histological image or input histological image segments) and output a pathological status (e.g., a pathological status metric) representing or relating to the level of chromosomal instability in the biological sample of the histological image (104). A variety of machine-learning, image processing, and data processing platforms may be used to implement the pathological status detection methods related to chromosomal instability described herein. Such methods may include Amazon Web Services (AWS) cloud computing services (e.g., using the P2 graphics processing unit (GPU) architecture), TensorFlow (an open-source program library for machine-learning), Apache Spark (an open-source, general-purpose distributed computing system used for big data analytics), Databricks (a web-based platform for working with Spark, that provides automated cluster management), Horovod (an open-source framework for distributed deep learning training using TensorFlow, Keras, PyTorch, and Apache MXNet), OpenSlide (a C library that provides an interface for reading whole-slide images), Scikit-Image (a collection of image processing methods), and Pyvips (a Python-based binding for the libvips image processing library).

The level of chromosomal instability in a biological sample of the training histological image may be high or low, as described herein. The trained machine-learning model may be used to characterize a disease in an individual (e.g., a patient) from which the biological sample of a histological image inputted into the machine-learning model is obtained, by computing a pathological status metric related to chromosomal instability of the input histological image.

### Example 2: Classifying Chromosomal Instability in a Patient Primary Tumor Sample

This example shows the deployment of the machine-learning model trained in Example 1 to classify the pathological status of a patient derived biological sample (e.g., a tumor sample, such as a tumor resection). This example further illustrates using the machine-learning model output to select a treatment based on the pathological status, related to chromosomal instability, of the biological sample (FIG. 3).

Biological samples are prepared for input into the machine-learning model from patient tumor samples (e.g., tumor resections, such as a tumor biopsy sample). Biological samples are stained, and fixed to a slide for histological imaging (e.g., input histological images). In some embodiments, the biological samples are stained with hematoxylin and eosin (H&E) stained or DAPI. Whole slide input histological images are captured at 10x magnifications. In some embodiments, the input histological images are subsectioned into 256 pixel x 256 pixel segments (301) using a sliding window approach, to generate input histological image segments. In some embodiments, the subsectioning of input histological images occurs in the machine-learning model to generate input histological image segments. The sliding window approach identifies segments of the whole slide input histological image with greyscale values greater than a threshold value. The threshold is chosen based on the greyscale value that differentiates the background image from the biological sample. The input histological image segments are inputted into the trained machine-learning model trained in Example 1.

The model uses the learned correlation between image features and matched chromosomal instability pathological metrics (e.g., fraction of the genome altered, “FGA”) to predict chromosomal instability of the image, such as binary high or low chromosomal instability (302). The predictions for all segments from the same biological sample are aggregated to classify the tumor sample’s pathological status (e.g., pathological status metric) that is related to chromosomal instability, such as binary high or low chromosomal instability (303).

A physician uses the pathological status of the biological sample as output from the machine-learning model to decide on a treatment regimen. For example, a treatment regimen may include an anti-chromosomal instability cancer therapy. The pathological status may also be used for additional clinical applications, such as to evaluate the effect of a treatment or diagnose cancer in a patient.

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

1. A method for characterizing a disease in a patient, comprising:
inputting one or more input histological images of a biological sample into a machine-learning model, wherein the machine learning model is trained using a plurality of training histological images and one or more matched chromosomal instability pathological metrics corresponding to the plurality of training histological images; and,
classifying a pathological status of the biological sample in the one or more input histological images using the trained machine-learning model.

2. The method of claim 1, wherein the biological sample comprises at least a portion of a solid tumor.

3. (canceled)

4. The method of claim 1, wherein the biological sample relates to a plurality of training or input histological images from the same patient.

5. The method of claim 1, wherein the one or more matched chromosomal pathological metrics are obtained from DNA from the biological sample of the training histological image.

6. The method of claim 1, wherein the one or more matched chromosomal pathological metrics are computed from DNA from the same patient as the biological sample of the training histological image.

7. The method of claim 1, wherein the one or more matched chromosomal pathological metrics and the biological sample of the training histological image come from the same patient.

8. The method of claim 1, wherein the one or more input histological images and/or the plurality of training histological images is:
i) captured between 2.5 x and 20 x magnification; and/or
ii) captured at a resolution between 256 pixels x 256 pixels and 10,000 pixels x 10,000 pixels.

9. (canceled)

10. The method of claim 1, wherein the one or more input histological images and/or the plurality of training histological images are hematoxylin and eosin (H&E) and/or 4′,6-diamidino-2-phenylindole (DAPI) stained images.

11. The method of claim 1, further comprising segmenting one or more whole images into a plurality of image tiles, wherein the image tiles are inputted into the machine-learning model as the input histological images and/or the training histological images.

12. The method of claim 1, wherein the machine-learning model segments the input histological images and/or the training histological images into tiles.

13. (canceled)

14. The method of claim 1, wherein the machine-learning model is an unsupervised model, a weakly-supervised model, or a human-in-the-loop model.

15-16. (canceled)

17. The method of claim 1, wherein the machine-learning model applies a model selected from the group consisting of Support Vector Machines (SVM), Random Forests (RF), Artificial Neural Network (ANN), Convolutional Neural Network (CNN), K-means, ResNet, DenseNet, and eXtreme Gradient Boosting (XGBoost).

18. The method of claim 1, wherein the one or more matched chromosomal instability pathological metrics and the training histological images are used to predict the pathological status of the input histological images.

19. The method of claim 18, wherein the pathological status is described as a metric, wherein the pathological status metric is selected from the group consisting of a probability of high chromosomal instability in the image, a continuous chromosomal instability score, and a binary classification of high or low chromosomal instability.

20-21. (canceled)

22. The method of claim 1, wherein the characterizing a disease comprises diagnosing the disease, informing a treatment strategy, evaluating the disease progression, predicting the disease prognosis, evaluating the effect of a treatment, or identifying a patient population for treatment.

23-27. (canceled)

28. The method of claim 1, wherein the disease is a cancer.

29. (canceled)

30. A system for characterizing a disease in a patient with machine-learning, comprising: one or more processors; a memory; and one or more programs with instructions for:
receiving data representing one or more input histological images of a biological sample; and,
classifying a pathological status of the biological sample in the one or more input histological images using a trained machine-learning model trained using a plurality of training histological images and one or more matched chromosomal instability pathological metrics corresponding to the plurality of training histological images.

31-59. (canceled)

60. A method for training a machine-learning model to analyze histological images of biological samples, comprising:
obtaining a chromosomal instability pathological metric for each training histological image of a plurality of training histological images; and
training the machine-learning model based on the plurality of training histological images and the matched chromosomal instability pathological metrics,
wherein the machine-learning model is trained to receive one or more input histological images and output a pathological status of the one or more input histological image.

61-84. (canceled)

85. A system for training a machine-learning model to predict a pathological status, comprising one or more processors; a memory; and one or more programs with instructions for:
receiving a plurality of chromosomal instability pathological metrics for a plurality of training histological images by calculating the chromosomal instability pathological metrics in the plurality of training histological images;
training the machine-learning model based on the plurality of training histological images and the matched chromosomal instability pathological metrics, wherein the machine-learning model is trained to receive one or more input histological images and output a pathological status of the one or more input histological images.

86-109. (canceled)

110. A non-transitory computer-readable storage medium storing one or more programs, the one or more programs comprising instructions, which when executed by one or more processors of an electronic device having a display, cause the electronic device to:
receive one or more input histological images of a biological sample;
input one or more input histological images of a biological sample into a machine-learning model trained using a plurality of training histological images and one or more matched chromosomal instability pathological metrics corresponding to the plurality of training histological images; and,
classify a pathological status of the biological sample in the one or more input histological images using the training machine-learning model.

111-134. (canceled)
